SEQUENCE LISTING (48 sequences)

SEQ ID NO: 1 - DNA, 22 bp, Artificial Sequence
  Tlr2 forward
gaagagtgagtggtgcaagtat

SEQ ID NO: 2 - DNA, 21 bp, Artificial Sequence
  Tlr2 reverse
aatgggctccagaagaatgag

SEQ ID NO: 3 - DNA, 21 bp, Artificial Sequence
  Tlr4 forward
tttcagctctgccttcactac

SEQ ID NO: 4 - DNA, 22 bp, Artificial Sequence
  Tlr4 reverse
gacaccacaacaatcacctttc

SEQ ID NO: 5 - DNA, 23 bp, Artificial Sequence
  MYD88 forward
ctgtgtctggtctattgctagtg

SEQ ID NO: 6 - DNA, 21 bp, Artificial Sequence
  MYD88 reverse
ttccttgctctgcaggtaatc

SEQ ID NO: 7 - DNA, 20 bp, Artificial Sequence
  IL1 forward
caaaggcggccaggatataa

SEQ ID NO: 8 - DNA, 23 bp, Artificial Sequence
  IL-1 reverse
ctagggattgagtccacattcag

SEQ ID NO: 9 - DNA, 22 bp, Artificial Sequence
  TNF forward
gcaggtctactttgggatcatt

SEQ ID NO: 10 - DNA, 19 bp, Artificial Sequence
  TNF reverse
agaagaggttgagggtgtc

SEQ ID NO: 11 - DNA, 22 bp, Artificial Sequence
  RelA forward
ctgtcctttctcatcccatctt

SEQ ID NO: 12 - DNA, 20 bp, Artificial Sequence
  RelA reverse
tcctctttctgcaccttgtc

SEQ ID NO: 13 - DNA, 21 bp, Artificial Sequence
  RelB forward
ctgcggatttgccgaattaac

SEQ ID NO: 14 - DNA, 23 bp, Artificial Sequence
  RelB reverse
acaccactgatatgtcctctttc

SEQ ID NO: 15 - DNA, 21 bp, Artificial Sequence
  Ccl2 forward
tcatagcagccaccttcattc

SEQ ID NO: 16 - DNA, 24 bp, Artificial Sequence
  Ccl2 reverse
ctctgcactgagatcttcctattg

SEQ ID NO: 17 - DNA, 24 bp, Artificial Sequence
  Ccl3 forward
ggcagattccacagaatttcatag

SEQ ID NO: 18 - DNA, 21 bp, Artificial Sequence
  Ccl3 reverse
tcgcttggttaggaagatgac

SEQ ID NO: 19 - DNA, 24 bp, Artificial Sequence
  IL10 forward
tccttgctggaggactttaagggt

SEQ ID NO: 20 - DNA, 24 bp, Artificial Sequence
  IL10 reverse
tgtctgggtcttggttctcagctt

SEQ ID NO: 21 - DNA, 21 bp, Artificial Sequence
  18s forward
cacggacaggattgacagatt

SEQ ID NO: 22 - DNA, 21 bp, Artificial Sequence
  18s reverse
gccagagtctcgttcgttatc

SEQ ID NO: 23 - DNA, 23 bp, Artificial Sequence
  GAPDH forward
ggtgtgaaccatgagaagtatga

SEQ ID NO: 24 - DNA, 22 bp, Artificial Sequence
  GADPH reverse
gagtccttccacgataccaaag

SEQ ID NO: 25 - DNA, 20 bp, Artificial Sequence
  granulocyte (Csf3) forward
tgttcccaaactgggttctt

SEQ ID NO: 26 - DNA, 18 bp, Artificial Sequence
  granulocyte (Csf3) reverse
tggctgccactgtttctt

SEQ ID NO: 27 - DNA, 20 bp, Artificial Sequence
  GM-CSF forward
agctctgaatccagcttctc

SEQ ID NO: 28 - DNA, 21 bp, Artificial Sequence
  GM-CSF reverse
ccacatctcttggtcccttta

SEQ ID NO: 29 - DNA, 21 bp, Artificial Sequence
  Ccl2 forward murine
ctcacctgctgctactcattc

SEQ ID NO: 30 - DNA, 21 bp, Artificial Sequence
  Ccl2 reverse
actacagcttctttgggacac

SEQ ID NO: 31 - DNA, 21 bp, Artificial Sequence
  Ccl3 forward murine
tcactgacctggaactgaatg

SEQ ID NO: 32 - DNA, 24 bp, Artificial Sequence
  Ccl3 reverse
cagcttataggagatggagctatg

SEQ ID NO: 33 - DNA, 20 bp, Artificial Sequence
  Rela forward murine
ccgacttgtttgggtgatct

SEQ ID NO: 34 - DNA, 20 bp, Artificial Sequence
  Rela reverse
tccgtctccaggaggttaat

SEQ ID NO: 35 - DNA, 20 bp, Artificial Sequence
  Relb forward murine
tgccgaatcaacaaggagag

SEQ ID NO: 36 - DNA, 20 bp, Artificial Sequence
  Relb reverse
tgctgaacaccacggatatg

SEQ ID NO: 37 - DNA, 22 bp, Artificial Sequence
  Tlr2 forward murine
cactatccggaggttgcatatc

SEQ ID NO: 38 - DNA, 22 bp, Artificial Sequence
  Tlr2 reverse
ggaagaccttgctgttctctac

SEQ ID NO: 39 - DNA, 20 bp, Artificial Sequence
  Tlr4 forward murine
gagcaaacagcagaggaaga

SEQ ID NO: 40 - DNA, 21 bp, Artificial Sequence
  Tlr4 reverse
ccaggtgagctgtagcattta

SEQ ID NO: 41 - DNA, 20 bp, Artificial Sequence
  MYD 88 forward murine
agcaactaggactgcctttc

SEQ ID NO: 42 - DNA, 22 bp, Artificial Sequence
  MYD88 reverse
gaactcttccactcagctatcc

SEQ ID NO: 43 - DNA, 23 bp, Artificial Sequence
  IL-1 forward murine
gcactacaggctccgagatgaac

SEQ ID NO: 44 - DNA, 24 bp, Artificial Sequence
  IL-1 reverse
ttgtcgttgcttggttctccttgt

SEQ ID NO: 45 - DNA, 21 bp, Artificial Sequence
  TNF forward murine
ttgtctactcccaggttctct

SEQ ID NO: 46 - DNA, 23 bp, Artificial Sequence
  TNF reverse
gaggttgactttctcctggtatg

SEQ ID NO: 47 - DNA, 20 bp, Artificial Sequence
  GADPH forward murine
ttgggttgtacatccaagca

SEQ ID NO: 48 - DNA, 20 bp, Artificial Sequence
  GADPH reverse
caagaaacaggggagctgag

(19) United States
(12) Patent Application Publication
(10) Pub. No.: US 2024/0092886 A1
(43) Pub. Date: Mar. 21, 2024

(54) MODULATING INTERLEUKIN-10 SIGNALING TO BOOST HEALING IN DIABETIC WOUNDS

(71) Applicant: Rush University Medical Center, Chicago, IL (US)

(72) Inventors: Sasha H. SHAFIKHANI, Elmwood Park, IL (US); Ruchi ROY, Chicago, IL (US)

(73) Assignee: Rush University Medical Center, Chicago, IL (US)

(21) Appl. No.: 17/767,620

(22) PCT Filed: Oct. 9, 2020
(86) PCT No.: PCT/US2020/055024

§ 371 Date: Apr. 8, 2022

Related U.S. Application Data
(60) Provisional application No. 62/912,765, filed on Oct. 9, 2019.

(51) Int. Cl. C07K 16/24; A61K 9/00; A61P 17/02

(52) U.S. Cl. CPC C07K 16/244; A61K 9/0014; A61P 17/02; A61K 2039/505; C07K 2317/76

(57) ABSTRACT

Methods of modulating IL-10 signaling in a diabetic patient are provided. The methods include administering a therapeutically effective amount of an agent that inhibits Interleukin-10 (IL-10) signaling to a wound site. The agent may antagonize IL-10 or IL-10R to inhibit IL-10 signaling. In some aspects the agent is an antibody or antigen-binding fragment that specifically binds to IL-10 or IL-10R.

### RELATED APPLICATIONS

This application claims the benefit of the filing date of U.S. Provisional Patent Application No. 62/912,765, filed Oct. 9, 2019, the contents of which is hereby incorporated by reference.

### FEDERALLY SPONSORED RESEARCH OR DEVELOPMENT

This invention was made with government support under grant number RO1 DK107713 awarded by the National Institutes of Health. The government has certain rights in the invention.

### STATEMENT REGARDING SEQUENCE LISTING

The Sequence Listing associated with this application is provided in text format in lieu of paper copy, and is hereby incorporated by reference into the specification. The name of the text file containing the Sequence Listing is “42960-326929 sequence listing_ST25”. The text file is 10 KB, was created on Oct. 8, 2020, and is being submitted electronically via EFS-Web, concurrent with the filing of this specification.

### BACKGROUND

### 1. Technical Field

The present disclosure pertains to methods and compositions for enhancing infection control and stimulating healing in diabetic wounds, in particular by targeting Interleukin-10 (IL-10).

### 2. Background Information

Previous reports have shown that macrophage response is delayed in diabetic wounds due to inadequate expression of pro-inflammatory cytokine C—C Motif Chemokine Ligand 2 (CCL2). Topical treatment with CCL2 significantly improves healing by jumpstarting the macrophage response. What remained unknown was why the expression of CCL2 is reduced in diabetic wounds early after injury.

In this disclosure, it is reported that the expression of pro-inflammatory cytokines is globally downregulated in diabetic wounds early after injury. The data demonstrates that reduction in production of pro-inflammatory cytokines is due to substantial downregulation in the expression of the toll-like receptors (TLRS). The TLRs are the major factories for the production of pro-inflammatory cytokines in wounds. The data reported here further shows that the reduction in TLR signaling is due to substantial rise in the level of IL-10 anti-inflammatory cytokine in diabetic wounds early after injury. IL-10 has been shown to dampen inflammatory responses by downregulating the expression of TLRs.

Methods and compositions for inhibiting IL-10 signaling to improve infection control and to stimulate healing in diabetic wounds are disclosed herein.

### BRIEF SUMMARY

Methods of modulating Interleukin-10 (IL-10) signaling in a diabetic patient are provided. The methods include administering a therapeutically effective amount of an agent that inhibits Interleukin-10 (IL-10) signaling to a wound site. The agent may antagonize IL-10 or IL-10R to inhibit IL-10 signaling.

### BRIEF DESCRIPTION OF THE DRAWINGS

FIG. 1A-F. Toll-like receptor signaling is significantly diminished during the acute phase wound healing in diabetic wound and in response to short-term exposure to high glucose.

C57B and db/db wounds tissues were collected 24h after wounding and assessed for the expression of indicated genes by Western blotting (a), by mRNA analysis using RT-PCR (b), and by immunohistochemistry (II-1C) (c-d). Representative regions at 40× and 400× magnification are shown in (c) and the corresponding data in (d). (N=5 mice/group, ≥9 random fields/wound/mouse). Black scale bars=500 μm; Red scale bars=200 μm. (e-f) hPBMCs exposed to 90 mg/dl or 300 mg/dl for 1 h were assessed for the expression of indicated genes by mRNA analysis using RT-PCR (e) or by Western blotting (f). Data are plotted as mean±SEM. Each experiment was repeated at least 2 times for (e) and at least 3 times for (f). *p<0.05, **p<0.01, ***p<0.001, Student's t-test.

FIG. 2A-H. IL-10 expression is significantly increased during the acute phase of healing in diabetic wounds and in response to short-term exposure to high glucose. (a-d) C57B and db/db day 1 wounds were assessed for IL10 levels by ELISA (a), by mRNA using RT-PCR (b), and for protein assessment by Western blotting (c) and corresponding densitometer of Western blot is shown in (d). (e-h) hPBMCs were exposed to 90 or 300 mg/dl glucose for 1 h and analyzed for IL10 expression by mRNA (e), or by protein level using Western blotting (f-g), or for glucose uptake using 2-NBDG assay (h). Data are plotted as the mean±SEM. (I≥3 mice/group (a-d); for (e-h), each experiment was done in triplicates and at 3 independent times; *p<0.05, **p<0.01, ***p<0.001, Student's t-test).

FIG. 3A-D. Blocking IL-10 signaling enhances TLR signaling and pro-inflammatory cytokines' production in PBMCs in the presence of high glucose. (a-d) hPBMCs were exposed to high glucose (300 mg/dl) for 1 h in the presence of either anti-IL-10 antibody (α-IL-10) or anti-IgG control isotype antibody (α-IgG) at 5 μg/ml, and assessed for glucose uptake in (a), for mRNA levels of indicated genes using RT-PCR (b), or by protein analyses using Western blotting (c) and corresponding densitometer tabulated data for Western blots are in (d). All data are plotted as the mean±SEM. (N>3 for (a); each experiment was repeated at least 2 times for (b) and at least 3 times for (c & d); *p<0.05, **p<0.01, ***p<0.001, Student's t-test).

FIG. 4A-D. Blocking IL-10 signaling enhances TLR signaling and pro-inflammatory cytokines production in diabetic wound. (a-g) db/db wounds were treated topically with α-IgG or α-IL10 antibodies (10 μg/wound), immediately after wounding. Wound tissues were analyzed for the expression of indicated genes by Western blotting (a), for mRNA levels by RT-PCR (b), by IHC (c), and by ELISA (e-g). (N=5 mice/group for all experiments. For (c-d)>9 random fields/wound/mouse. Data were then normalized per wound area and plotted as mean±SEM, *p<0.05, ** p<0.01, ***p<0.001, Student's t-test.)

FIG. 5A-G. Blocking IL-10 signaling enhances leukocyte and macrophage responses during the acute phase of wound healing. Immediately after wounding, diabetic (db/db) wounds were treated topically with α-IgG or α-IL10 antibodies. Wound tissues were harvested at indicated timepoints and assessed for their leukocytes contents by H&E staining (a), and for M1 and M2 macrophages, by IF microscopy using F4/80/iNOS or F4/80/MMR/CD206 respectively (c-d). The corresponding data are plotted as mean as mean±SEM and shown in (b, e, & f) respectively.

FIG. 6A-F. Blocking IL-10 signaling stimulates wound healing in diabetic wound. db/db wounds were treated with either α-IgG or α-IL10 antibodies and assessed for wound healing by digital photography (a-b); by the histochemical assessment of re-epithelization and epidermal thickening, using H&E staining (c & d); and by histochemical assessments of the Vimentin, α-SMA, Masson's Trichrome staining, and Elastin healing markers (e-g). Representative images are shown in (a, c, & e) and the corresponding data are plotted as mean as mean±SEM in (b, d, & f). Black scale bars=100 μm, red scale bars=200 μm. (N≥5 mice/group, >9 random fields/wound/mouse, *p<0.05, ** p<0.01, ***p<0.001, Student's t-test).

FIG. 7A-E. Pro-inflammatory cytokines production is reduced during the acute phase of wound healing early after injury. (a) Normal (C57B) and diabetic (db/db) wounds were collected at indicated time points after wounding (day 0) and assessed for their IL-β and TNF-α contents by ELISA. (b-c) C57B and db/db day 1 wounds were assessed for the indicated pro-inflammatory cytokines' gene expression by mRNA analyses using RT-PCR. (d) Day 1 normal and diabetic wounds were assessed for the indicated pro-inflammatory cytokines' protein levels by Western blotting. (e) Densitometer measurements of Western blots of the indicated pro-inflammatory cytokines are plotted as the mean±SEM. (N=5 mice per group. Statistical significance is denoted by asterisks, * p<0.05, ** p<0.01 and *** p<0.001. Student's t-test was applied for these analyses).

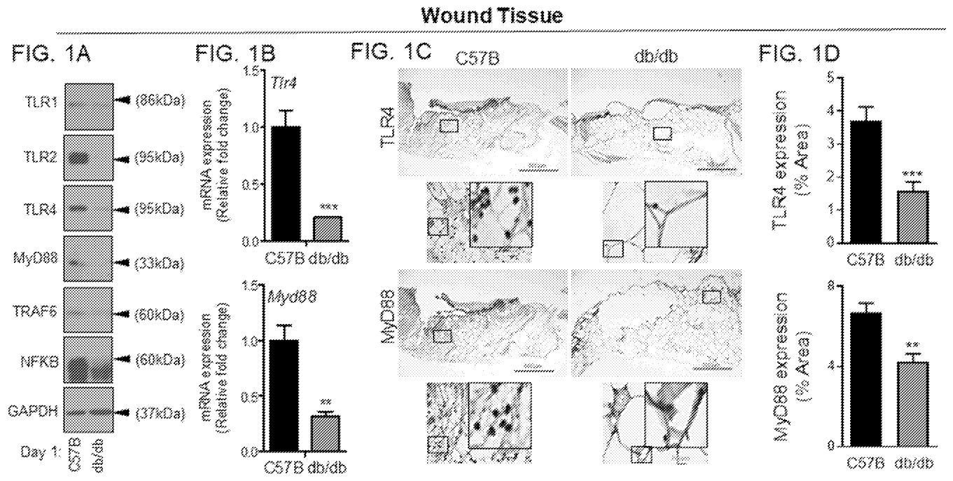

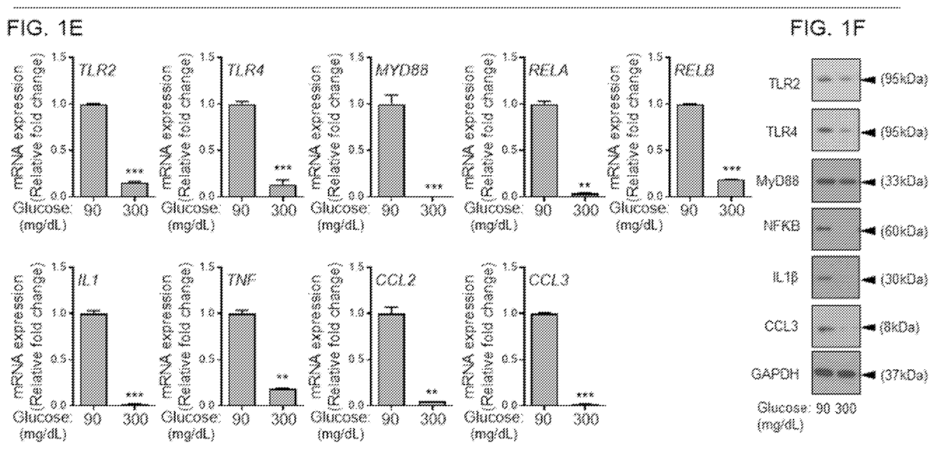

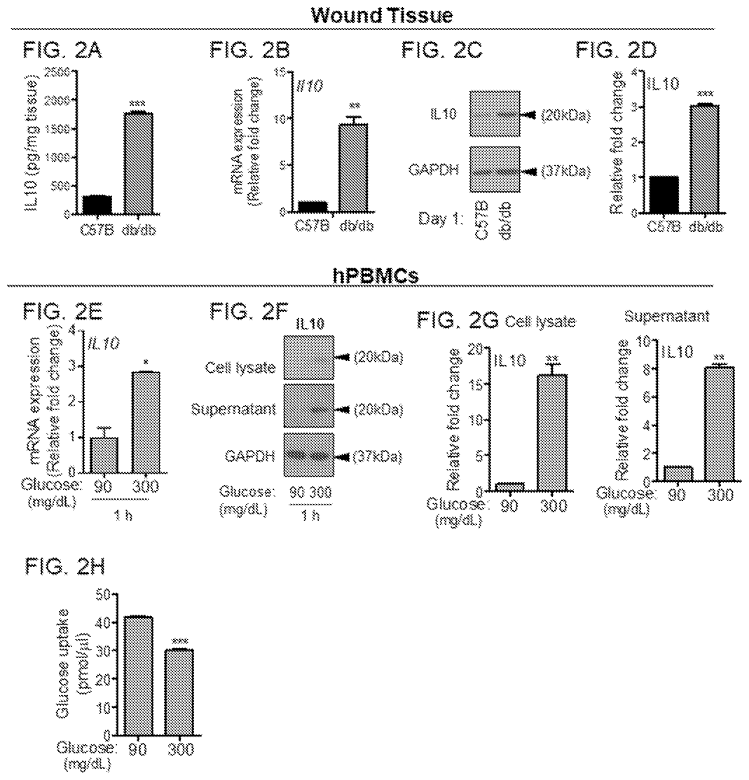

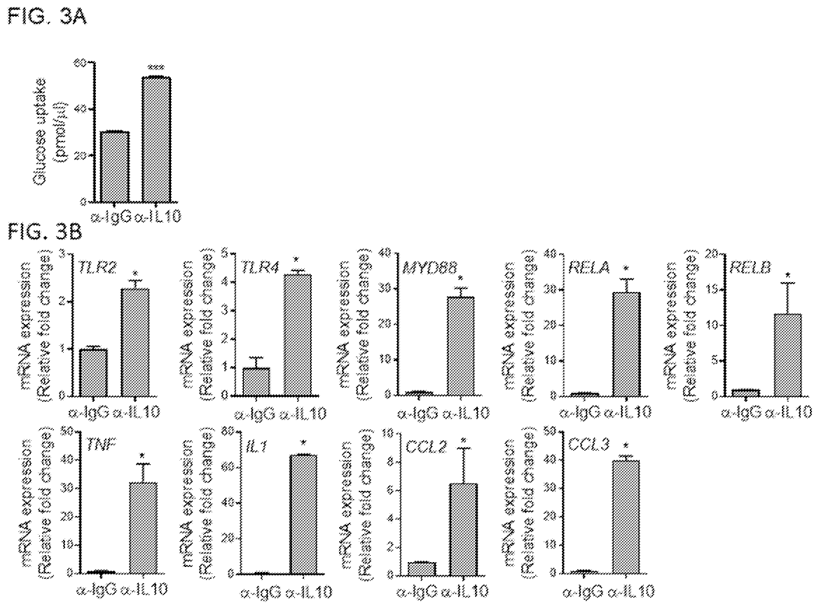

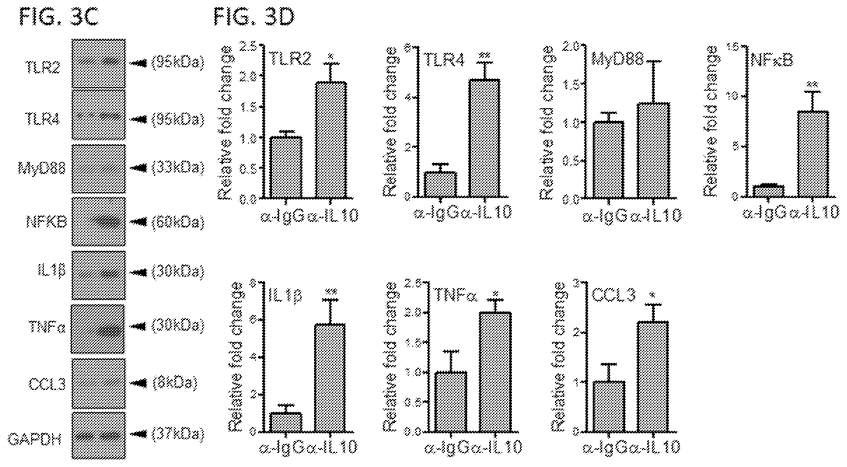

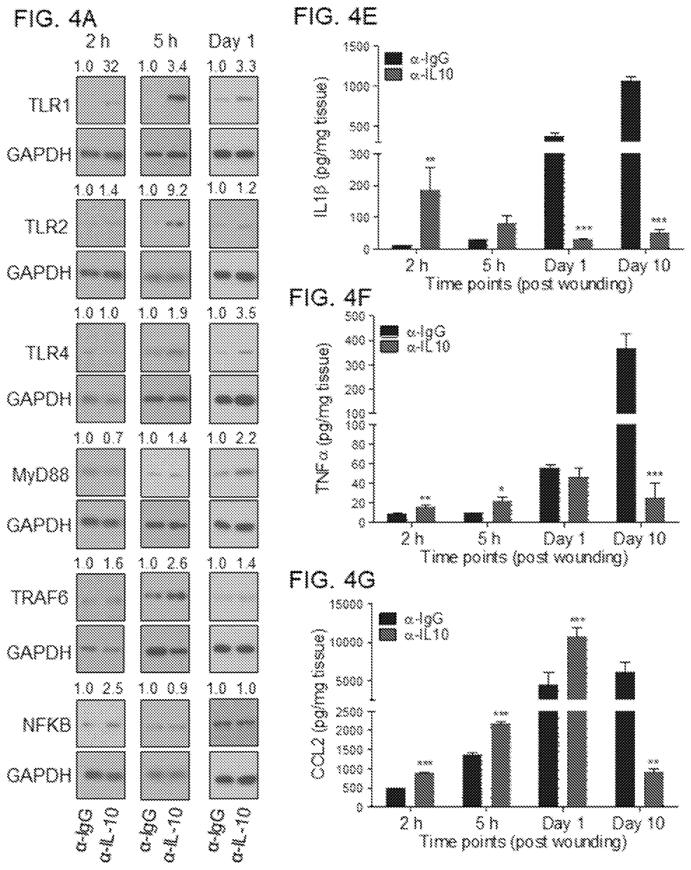

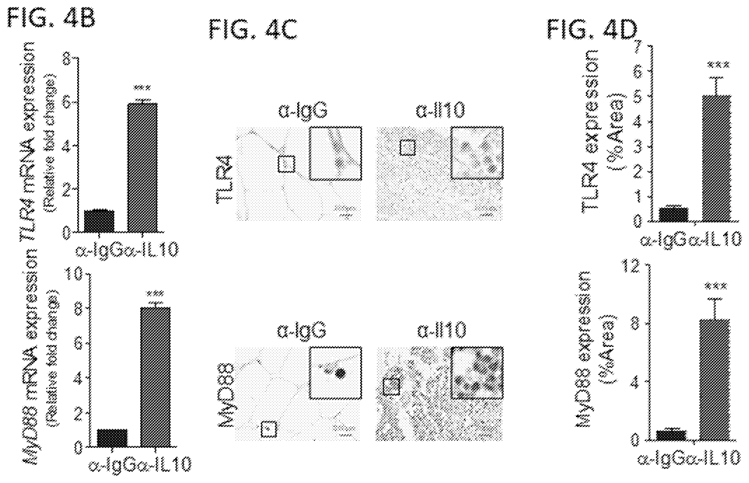

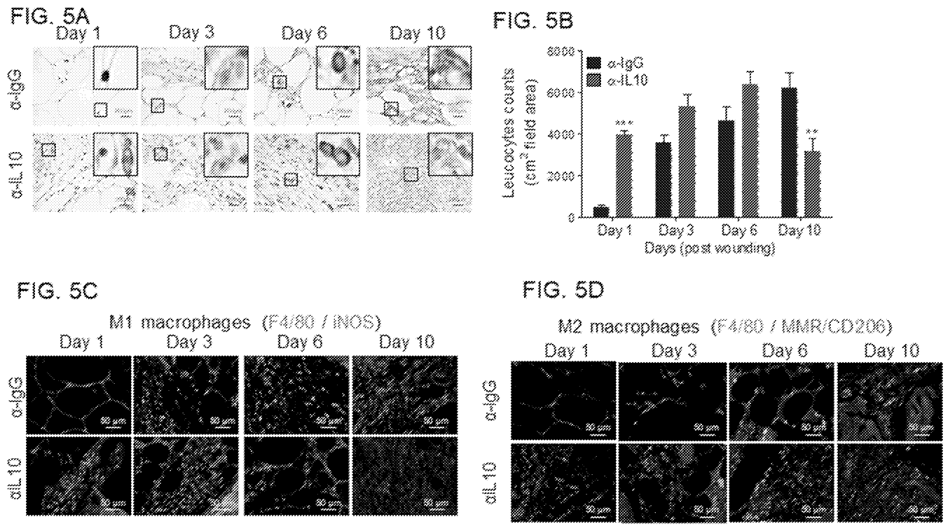

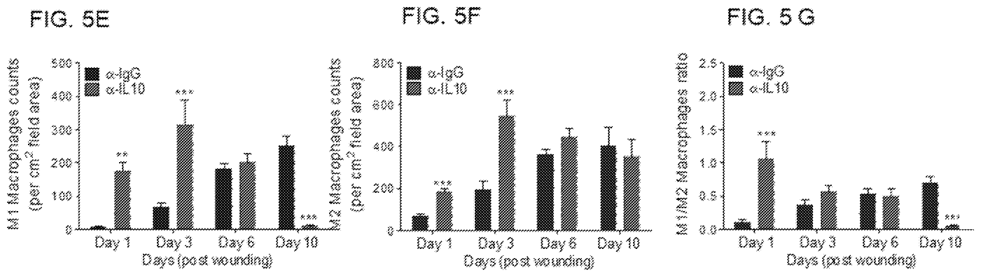

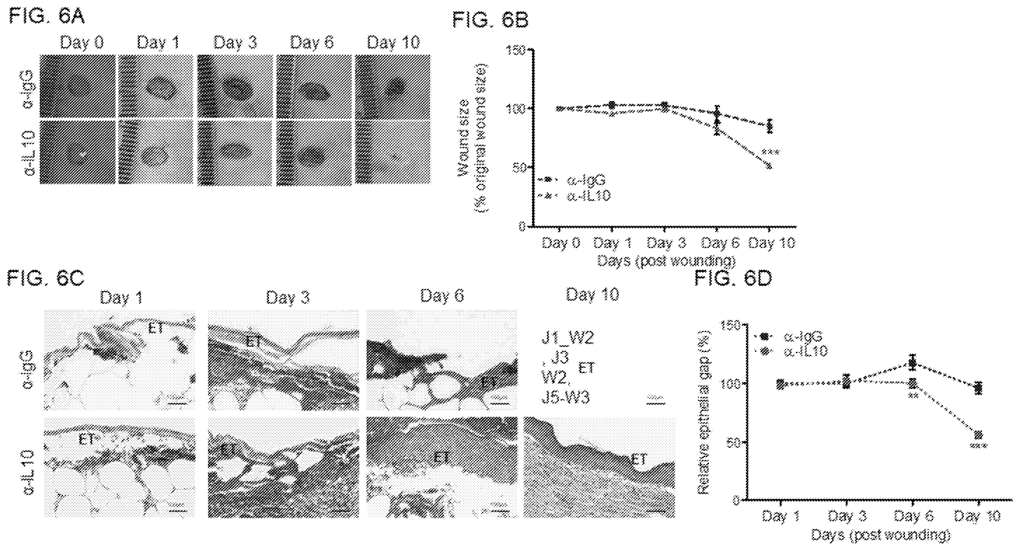

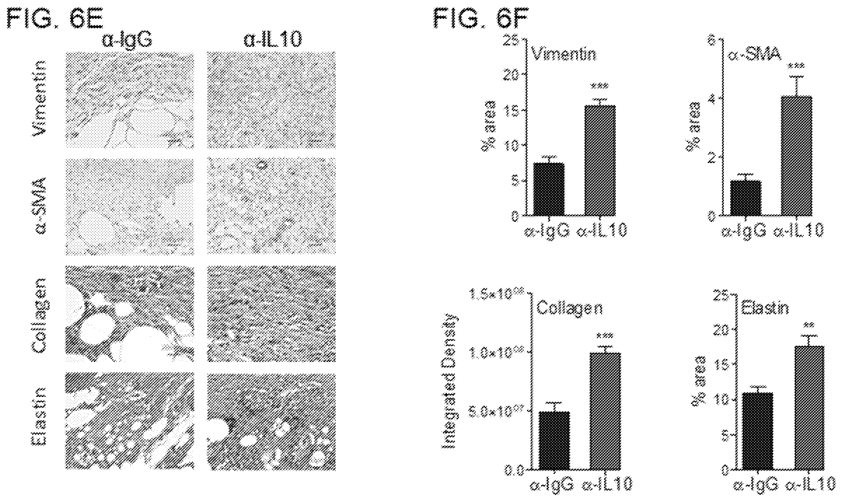

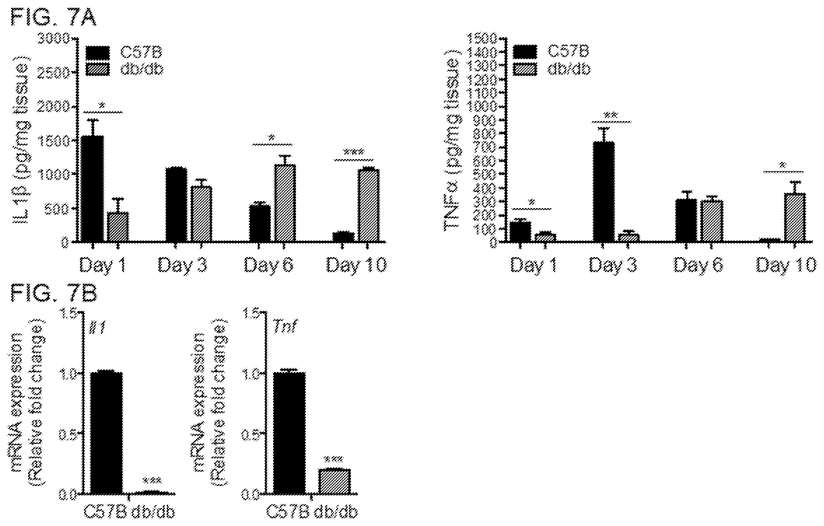

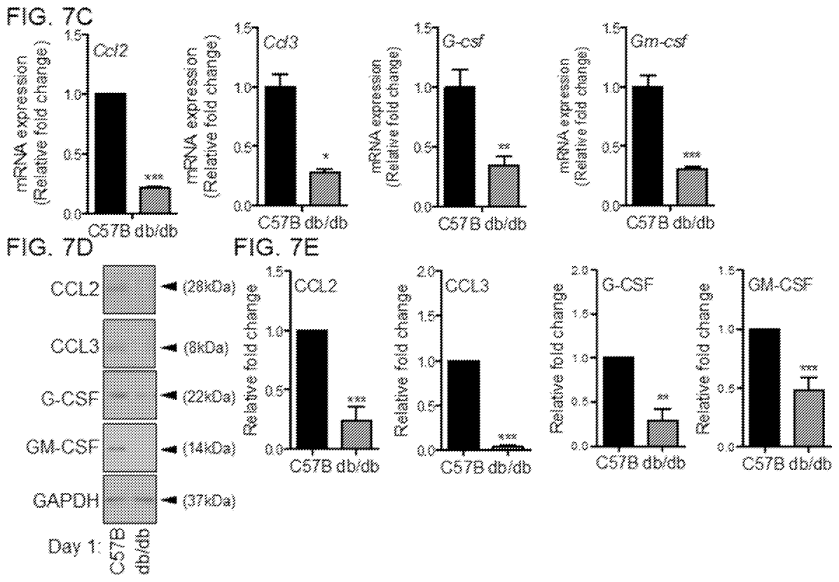

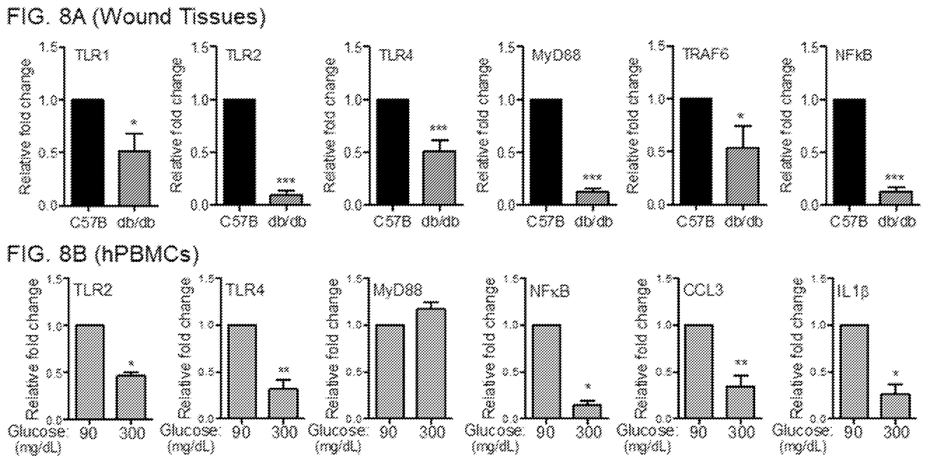

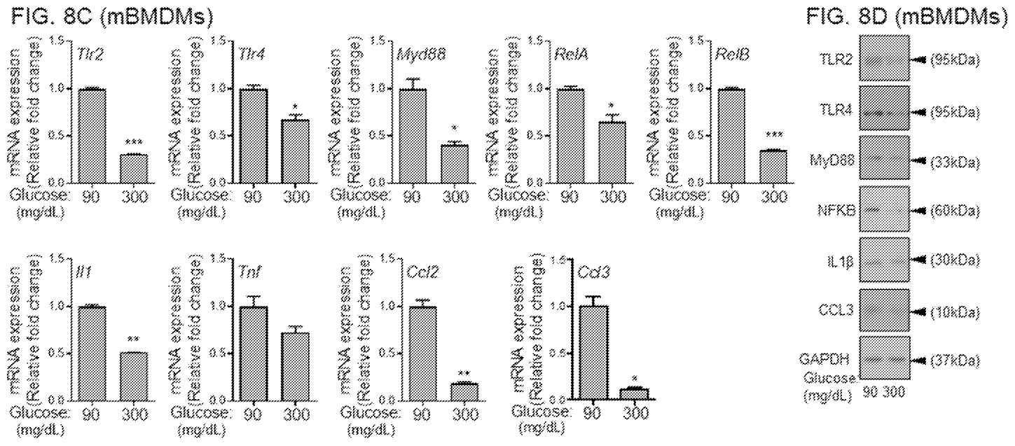

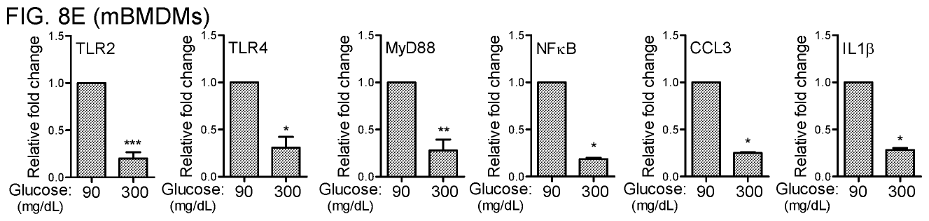

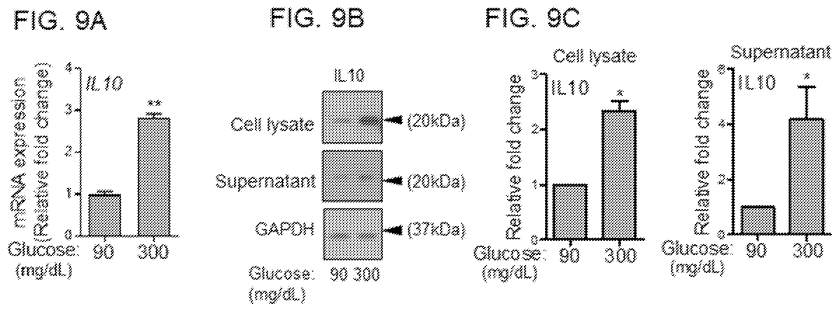

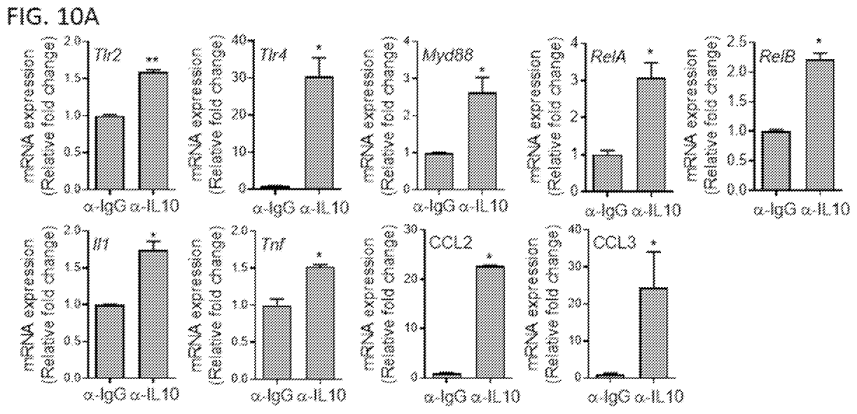

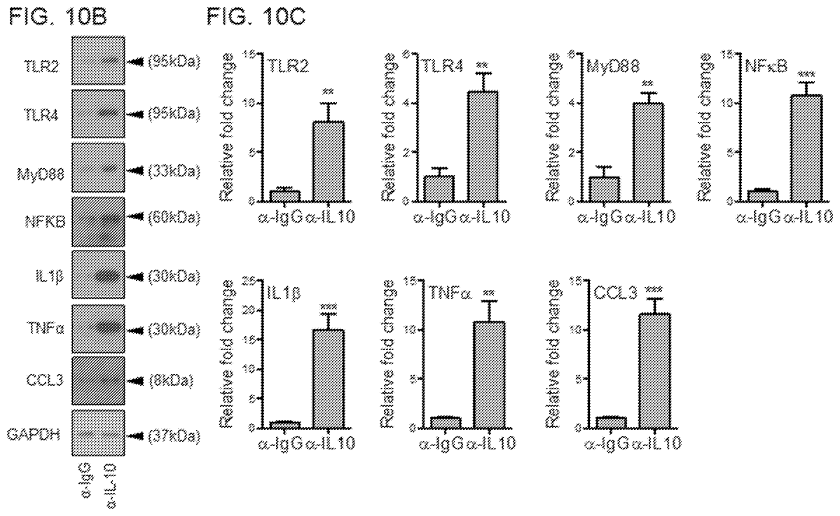

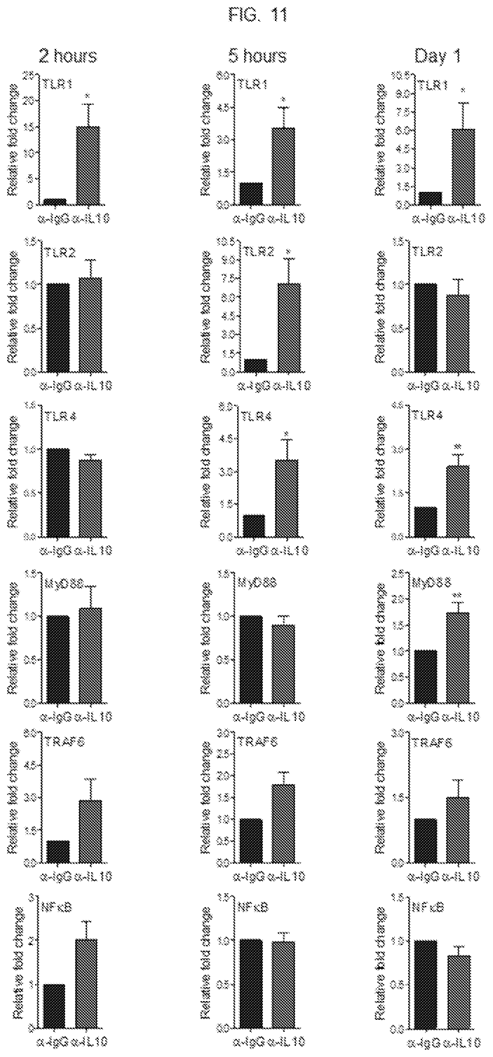

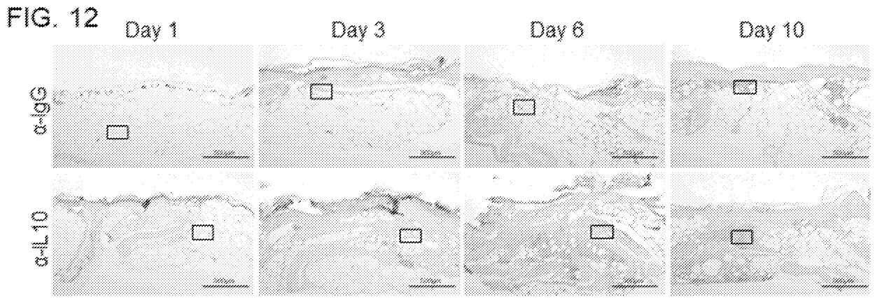

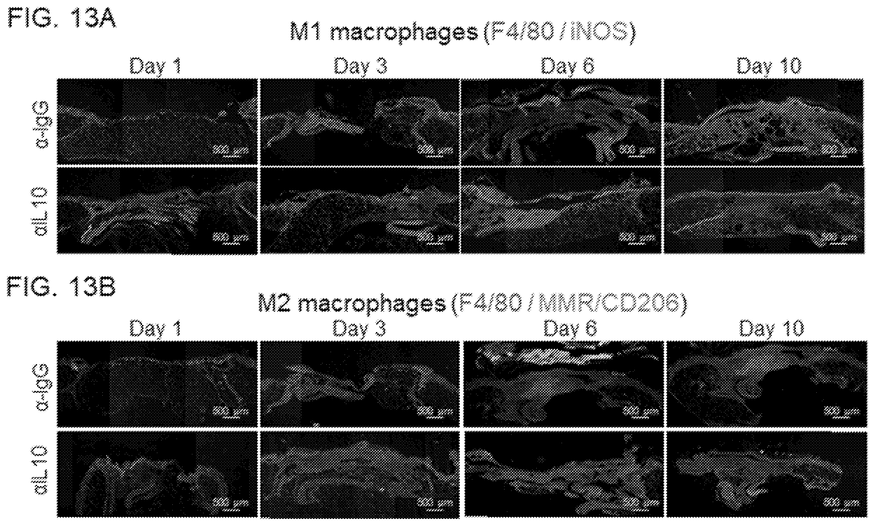

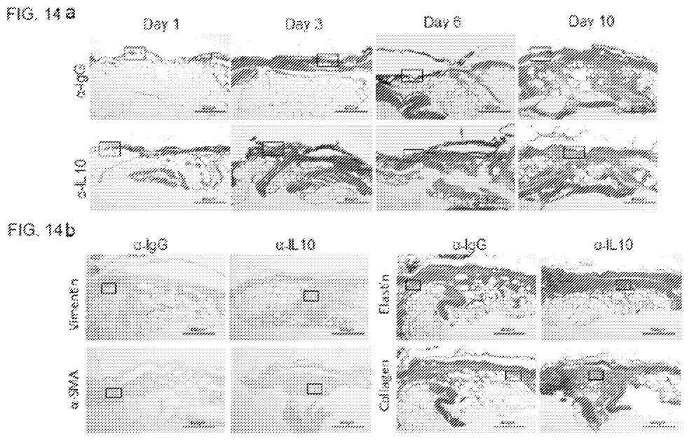

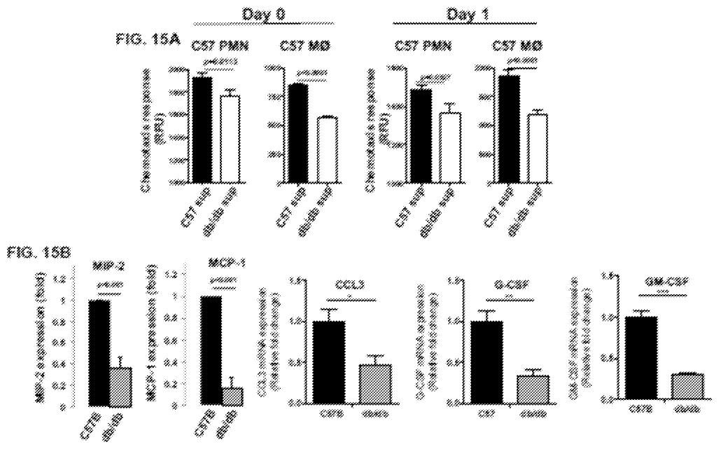

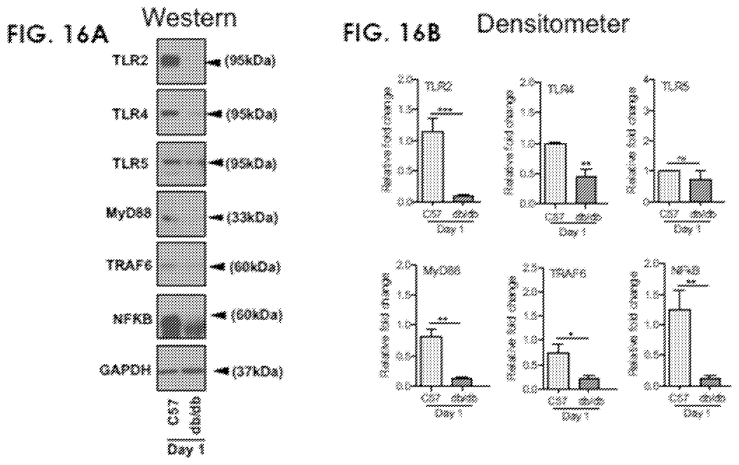

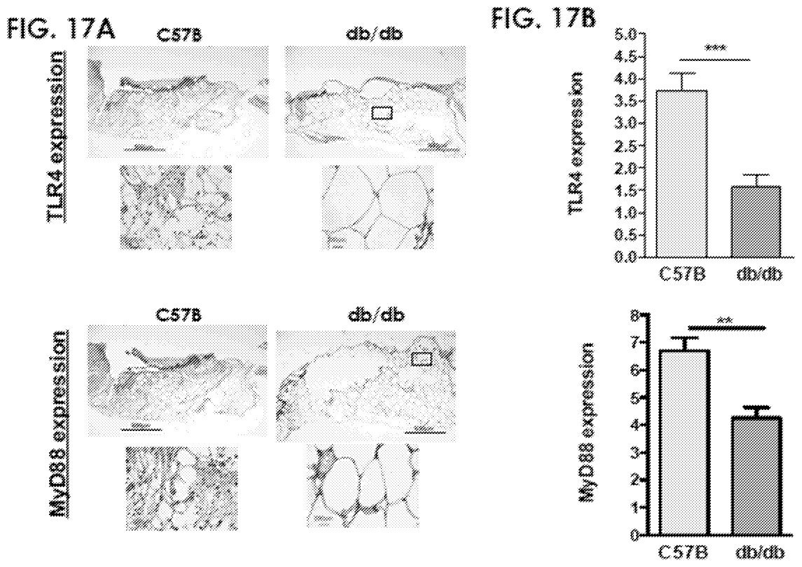

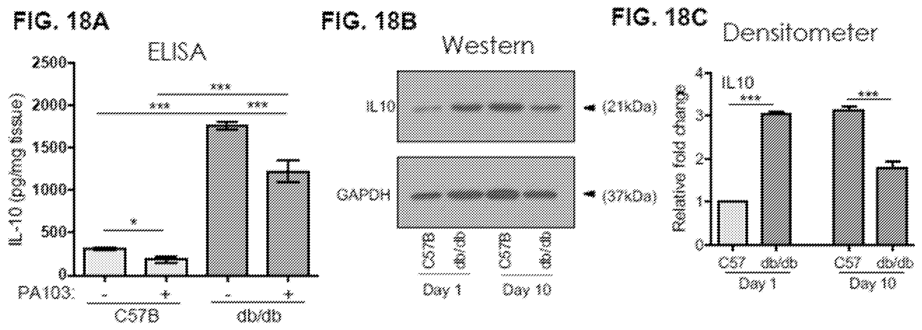

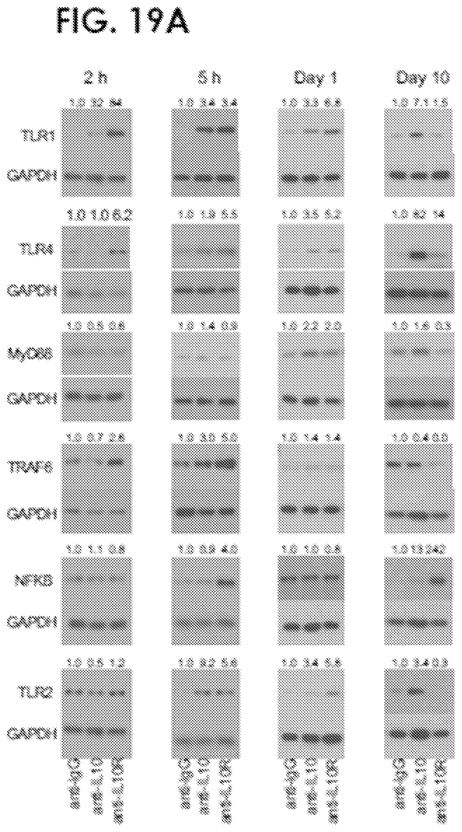

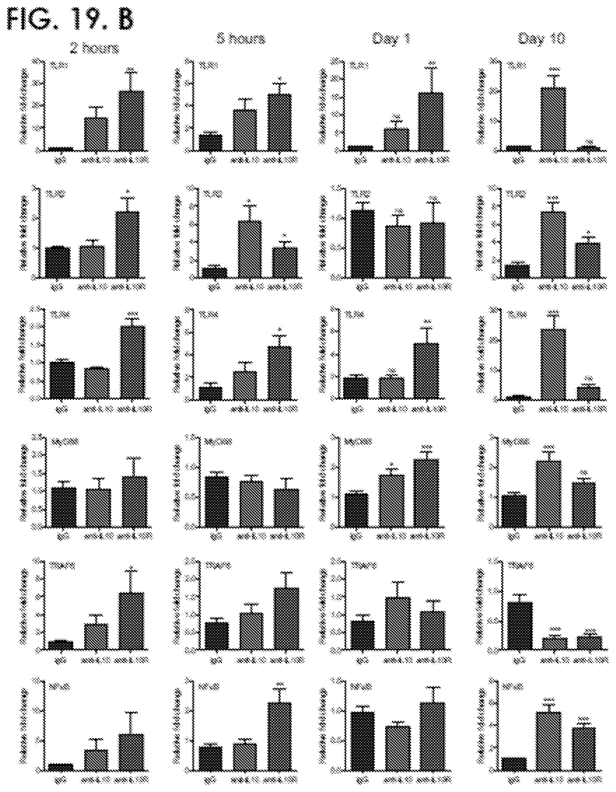

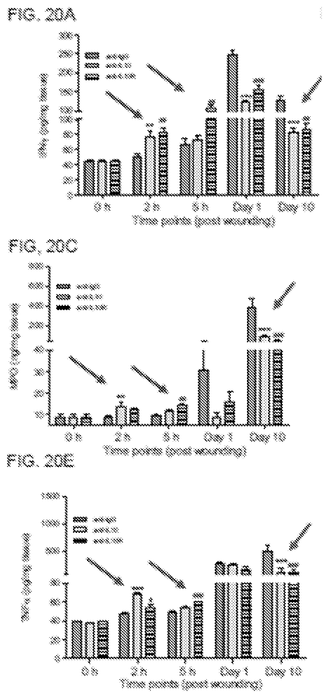

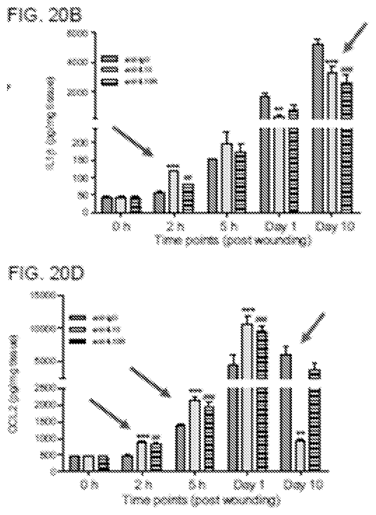

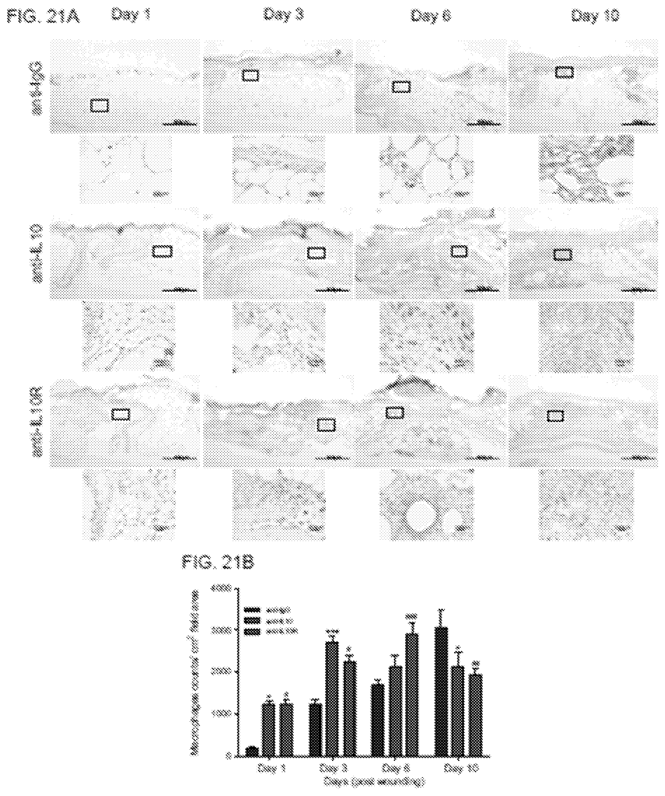

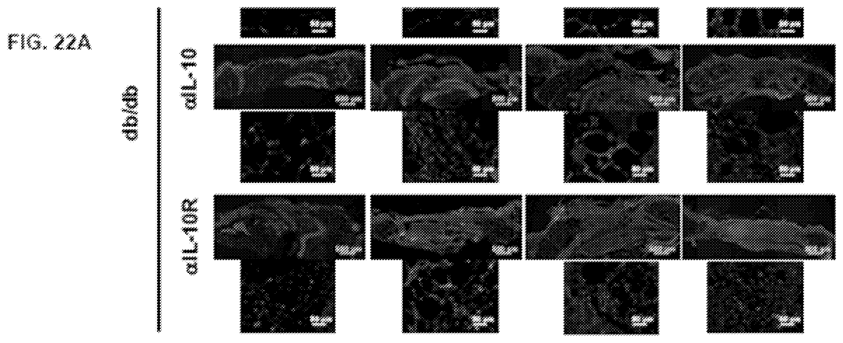

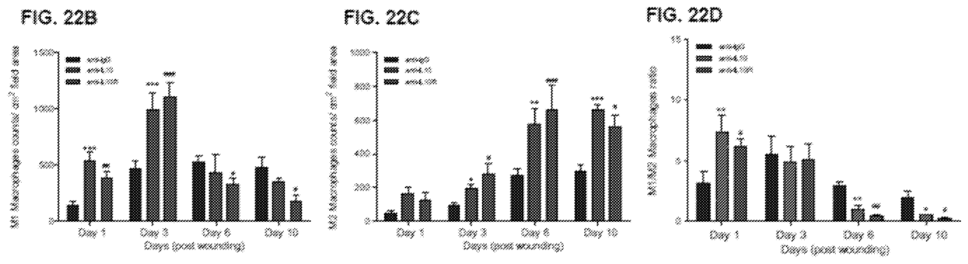

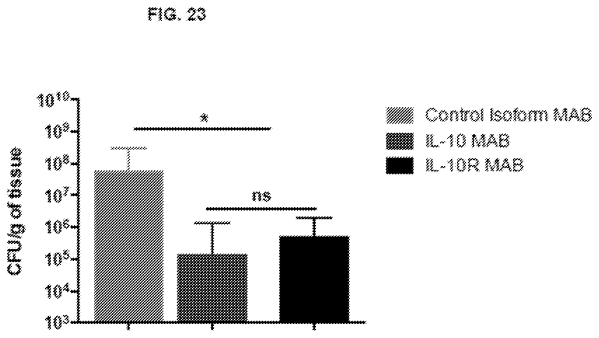

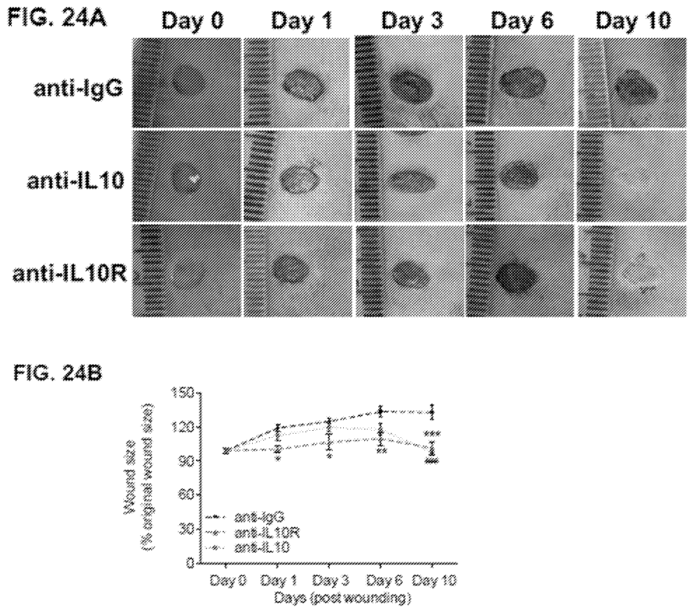

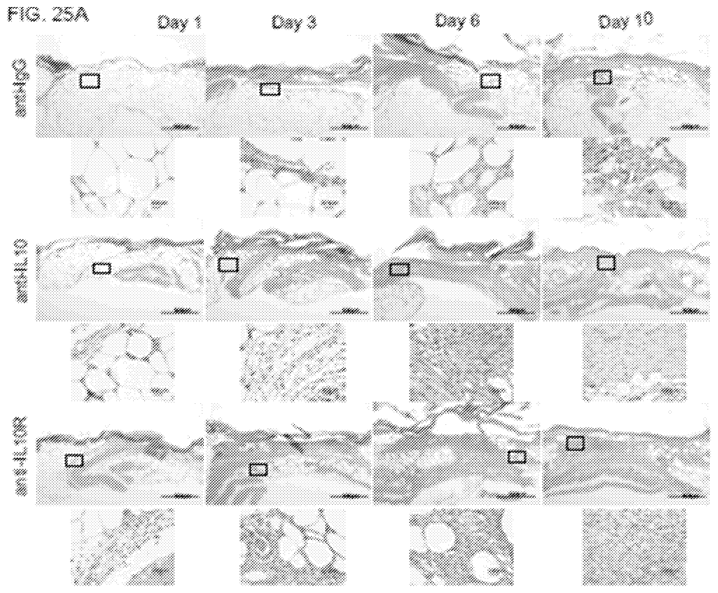

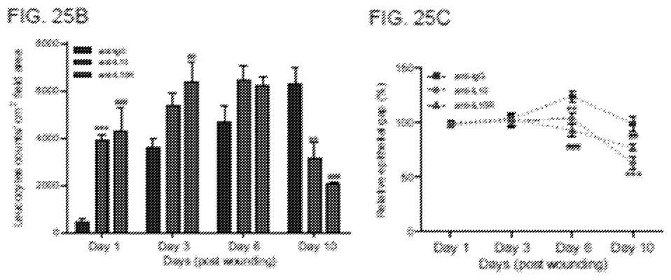

FIG. 8A-E. Toll-like receptor signaling is significantly diminished during the acute phase wound healing in diabetic wound and in response to acute exposure to high glucose. (a) Densitometric analyses related to Western blot data in FIG. 1a (N=5 mice/group). (b) Densitometric analyses related to Western blots in FIG. 1f. (c-e) mBMDMs were exposed to 90 mg/dl 300 mg/dl for 1 h and assessed for indicated genes by RT-PCR (c) or for protein levels by Western blotting (d). The corresponding densitometer data in (e). (Each experiment was repeated at least 2 times for (c) and at least 3 times for (d & e), all data shown as mean±SEM, *p<0.05, **p<0.01, ***p<0.001, Student's t-test).

FIG. 9A-C. Differential regulation of IL-10 and IL113 by short-term and long-term exposures to high glucose. (a-c) mBMDMs were exposed to 90 mg/dl or 300 mg/dl for 1h and assessed for IL-10 expression by mRNA analysis using RT-PCR (a), and for protein levels by Western blotting (b) and the densitometer measurements of Western blots are plotted in (c). (Each experiment was repeated at least 2 times for (a) and at least 3 times for (b-c). Data were plotted as mean±SEM. (N≤3 mice/group, *p<0.05, **p<0.01, ***p<0.001, Student's t-test).

FIG. 10A-C. Blocking IL10 signaling enhances TLR signaling and pro-inflammatory cytokines' production in BMDMs in the presence of high glucose. (a-c) mBMDMs were exposed to media containing high glucose (300 mg/dl) for 1 h in the presence of either α-IL-10 or α-IgG. The expression of indicated genes was assessed by mRNA analysis using RT-PCR (a), or by protein levels using Western blotting (b) and the corresponding densitometer measurements of Western blots are plotted as mean±SEM (c). (Each experiment was repeated at least 2 times for (a) and at least 3 times for (b and c); *p<0.05, **p<0.01, ***p<0.001, Student's t-test).

FIG. 11. Blocking IL10 signaling enhances TLR signaling and pro-inflammatory cytokines production in diabetic wound. Densitometer measurements of Western blots related to FIG. 4a) are plotted as the mean±SEM. (N=5 mice/group, *p<0.05, **p<0.01, ***p<0.001, Student's t-test).

FIG. 12. Blocking IL-10 signaling enhances leukocyte responses during the acute phase of wound healing. Full wound images associated with FIG. 5a are shown here. Black scale bars=500 μm.

FIG. 13-A-B. Blocking IL-10 signaling enhances leukocyte responses in diabetic wound. Full wound images associated with M1 (F4/80/iNOS) and M2 (F4/80/MMR/CD206) macrophage staining in FIG. 5c and d are shown in (a & b) respectively.

FIG. 14A-B. Blocking IL-10 signaling stimulates wound healing in diabetic wound. (a) Full wound images associated with FIG. 6c are shown here. (b) Histological images of the day 10 diabetic (db/db) full wounds which were treated with α-IgG or α-IL10 antibodies and stained with Vimentin, α-SMA, Elastin, and Collagen, healing markers at 40× magnification (see FIG. 6e). The square within each image represents the magnified region of the wound that were presented in FIG. 6c and FIG. 6e. Black scale bars are 500 μm.

FIG. 15A-B: Expression of Pro-inflammatory cytokines is significantly reduced in diabetic wounds early after injury. (A) Skin tissues from the normal and diabetic wound edges (1 mm from the rim) were harvested on day 0 (right after wounding) and on day 1 post wounding. These tissue explants were cultured and allowed to secrete chemokines into culture media. The supernatants from these cultures were then evaluated for their chemokine levels by assessing their ability to chemoattract normal neutrophil (C57 PMN) or macrophages (C57 Mø). The results are shown as the mean±SEM (n=18; 6 mice per group, each done in duplicates, *p<0.05, **p<0.01; ***p<0.001, Student's t-test). These results indicate that pro-inflammatory cytokines expression is globally reduced in diabetic wounds early after injury. (B) The expression levels of indicated pro-inflammatory cytokines in day 1 C57 and db/db wounds were determined by RT-PCR and the results are shown as the mean±SEM (n=4 mice per group). These results corroborate the data in (A).

FIG. 16A-B: Signaling through Pattern Recognition Receptors (PRRs) is significantly reduced in diabetic wounds early after injury. (A) Skin tissues from the normal (C57) and diabetic (db/db) wound edges (1 mm from the rim) were harvested on day 1 post wounding and assessed for the expression of the indicated PRR genes by Western blotting. (B) The corresponding densitometer measurements are plotted as the mean±SEM (n>3 mice per group, each done in duplicates, *p<0.05, **p<0.01; ***p<0.001, Student's t-test). These results indicate that signaling through PRR is significantly reduced in diabetic wound early after injury.

FIG. 17A-B: Signaling through Pattern Recognition Receptors (PRRs) is significantly reduced in diabetic wounds early after injury. (A) Skin tissues from the normal (C57) and diabetic (db/db) wound edges (1 mm from the rim) were harvested on day 1 post wounding and assessed for the expression of the indicated PRR genes by Immunohistochemistry (IHC). (B) The corresponding data are plotted as the mean±SEM (n>3 mice per group, 10 random fields per mouse, *p<0.05, **p<0.01, ***p<0.001, Student's t test). These results corroborate the data in FIG. 2.

FIG. 18A-C: IL-10 expression is significantly increased in infected and uninfected diabetic wounds early after injury. Normal (C57B) or diabetic (db/db) wounds were either infected with 1000 P. aeruginosa PA103 strain or treated with PBS. Wound tissues were harvested and assessed for the expression of IL-10 by: (A) ELISA 1 day post-infection; or (B) by Western blotting on day 1 and day 10 after infection. (C) Corresponding data for (B) are plotted as the mean±SEM (n=4 mice per group, *p<0.05, **p<0.01, ***p<0.001, Student's t-test). These results indicate that IL-10 expression is significantly higher in diabetic wounds early after injury.

FIG. 19A-B: Blocking IL-10 signaling restores the dynamics of PRRs in diabetic. Diabetic (db/db) wounds were either treated with anti-IL-10 or anti-IL-10R antibodies (10 μg/wound) or control anti-IgG isoform. (A) Wounds were harvested at indicated time points and assessed for the expression of the indicated PRR genes by Western blotting. (B) The corresponding densitometer measurements are plotted as the mean±SEM (n>3 mice per group, *p<0.05, **p<0.01, ***p<0.001, One-way ANOVA). Note, that blocking IL-10 signaling results in significant increases in the expression of the indicated PRRs, particularly early after treatment.

FIG. 20A-E: Blocking IL-10 increases the production of pro-inflammatory cytokines. Diabetic (db/db) wounds were either treated with anti-IL-10 or anti-IL-10R antibodies or control anti-IgG isoform. (10 μg/wound) (A) Wounds were harvested at indicated timepoints and assessed for the expression of the indicated pro-inflammatory cytokines by ELISA. The corresponding data are plotted as the mean±SEM (n=4 mice per group, *p<0.05, **p<0.01, ***p<0.001, One-way ANOVA). Note, that the expression of these pro-inflammatory cytokines are significantly higher in IL-10 blocked wounds as compared to the control groups early after injury but lower in day 10 wounds. Red arrows point to increases in the expression of pro-inflammatory cytokines in early diabetic wounds whereas the purple arrows point to lower expression in day 10 wounds.

FIG. 21A-B: Blocking IL-10 enhances the Me response in diabetic wounds. (A) Skin tissues from anti-IgG, anti-IL10 and anti-IL1OR treated diabetic (db/db) wounds were harvested at indicated time-points (day 1, day 3, day 6 and day 10) post wounding (day 0), fixed and stained for macrophages with F4/80. Black scale bar=500 μm for 4× magnification and red scale bar=200 μm for 40× magnification. (B) The corresponding macrophages counts are plotted as the mean±SEM. (n=5 mice per group, * p<0.05, ** p<0.01 and *** p<0.001, One-way ANOVA). Note that compared to the control group, blocking IL_10 significantly increases the Mø influx in diabetic wounds in Days 1 and 3 but decreases in Day 10.

FIG. 22A-D: Impact of IL-10 inhibition on the dynamics of Mø differentiation in diabetic wound. (A) Skin tissues from anti-IgG, anti-IL10 and anti-IL10R treated diabetic (db/db) wounds were harvested at indicated time-points (day 1, day 3, day 6 and day 10) post wounding (day 0), fixed and stained for M1 and Mø type macrophages with antibodies against iNOS (GREEN) and MMR/CD206 (RED) respectively. (Yellow scale bar=500 μm for 5× magnification and white scale bar=50 μm for 40× magnification). The impact of IL-10 inhibition on M1 Møs (B), M2 Møs (C), and the M1/M2 ratios (D) were plotted as the mean±SEM (n=5 mice per group, * p<0.05, ** p<0.01 and *** p<0.001. One-way ANOVA). Note that blocking IL-10 signaling results in increased M1 and M2 Møs but M1 Møs predominate in day 1 and day 3 wounds, whereas, M2 Møs predominate in day 6 and day 10 wounds.

FIG. 23: Blocking IL10 and its receptor stimulates infection control in diabetic mice. Diabetic (db/db) wounds were either treated with anti-IL-10 or anti-IL-10R antibodies or control anti-IgG isoform and infected with 1000 PA103 P. aeruginosa bacteria. Wounds were harvested 24 h post-infection (Day 1) and evaluated for their bacterial burden by CFU count. (n=3 mice per group. *p<0.05, One-way ANOVA). Note that blocking IL-10 signaling significantly stimulates infection control in diabetic wound.

FIG. 24A-B. Blocking IL10 and its receptor stimulates wound healing in diabetic wound. Wound healing in the mock and anti-IL10 and anti IL-10R-treated diabetic wounds was monitored by digital photography and wound areas were determined by tracing. Representative images from days 1, day 1, day 3, day 6, and 10 are shown in (A) and the tabulated results are shown as mean±SEM in (B) (n=6 mice per group, * p<0.05, ** p<0.01 and *** p<0.001. One-way ANOVA).

FIG. 25A-C. Blocking IL10 and its receptor does not lead to persistent inflammation and stimulates wound healing in diabetic wound. (A) Skin tissues from anti-IgG, anti-IL10 and anti-IL10R treated diabetic (db/db) wounds were harvested at indicated time-points (day 1, day 3, day 6 and day 10) post wounding (day 0), fixed and stained for hematoxylin and eosin (H&E). Black scale bar=500 μm for 4× magnification and red scale bar=200 μm for 40× magnification. (B) The number of leucocytes were determined and the corresponding data are plotted as the mean±SEM. Data were normalized per wound area. (n=4, 10 random fields per mice per group, * p<0.05, ** p<0.01.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings. Unless otherwise defined, all technical and scientific terms used herein have the same meaning as commonly understood by one of ordinary skill in the art to which this disclosure pertains. In case of conflict, the present document, including definitions, will control.

Methods of modulating Interleukin-10 (IL-10) signaling in a diabetic patient are provided. The methods include administering a therapeutically effective amount of an agent that inhibits IL-10 signaling to a wound site. The agent may antagonize IL-10 or IL-10R to inhibit IL-10 signaling. In some aspects the agent is an antibody or antigen-binding fragment that specifically binds to IL-10 or IL-10R.

As shown herein administration of an agent that inhibits IL-10 signaling leads to significant increases in toll-like receptor (TLR) signaling, pro-inflammatory cytokine production, and macrophage response in diabetic wounds early after injury.

An “agent that modulates Interleukin-10 (IL-10) signaling” can be but is not limited to, a nucleic acid, small molecule, peptide, or polypeptide (e.g., antibody). In some embodiments, IL-10 signaling may be inhibited. In some embodiments, the agent is an antibody. In some embodiments, the antibody inhibits IL-10 signaling. In some embodiments, the antibody is an anti-IL-10 antibody or an anti-IL10R antibody.

Inhibiting IL-10 signaling by topical application of an agent comprising antibodies or antigen-binding fragments thereof that specifically bind IL-10 or IL-10 receptor (IL-10R), leads to significant increases in TLR signaling, pro-inflammatory cytokine production, and macrophage response in diabetic wounds early after injury. Importantly, treatments with anti IL-10 or anti-IL-10R antibodies or antigen-binding fragments thereof does not result in persistent non-resolving inflammation. Rather, inflammation subsides over time as indicated by reduced pro-inflammatory cytokines and reduced inflammatory leukocytes in day 10 wounds. Interestingly, the macrophages drawn into the treated diabetic wounds early after injury are dominated by pro-inflammatory M1 macrophages but they switch to M2 reparative macrophages as the wound heals. Finally, inhibiting IL-10 activity also substantially improves the ability of diabetic wounds to control infection and exhibit significant improvement in healing.

The term “antibody” as used herein refers to whole antibodies that interact with (e.g., by binding, steric hindrance, stabilizing/destabilizing, spatial distribution) an IL-10 or IL-10R epitope and inhibit signal transduction. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and V_{L} regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the disclosure), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The phrase “antibody fragment”, as used herein, refers to one or more portions of an antibody that retain the ability to specifically interact with (e.g., by binding, steric hindrance, stabilizing/destabilizing, spatial distribution) an IL-10 or IL-10R epitope and inhibit signal transduction. Examples of binding fragments include, but are not limited to, a Fab fragment, a monovalent fragment consisting of the VL, VH, CL and CHI domains; a F(ab)₂ fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; a dAb fragment (Ward et al, (1989) Nature 341 :544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al, (1988) Science 242:423-426; and Huston et al, (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (VH-CH1-VH-CH1) which, together with complementary light chain polypeptides, form a pair of antigen binding regions (Zapata et al., (1995) Protein Eng. 8: 1057-1062; and U.S. Pat. No. 5,641,870).

The phrases “monoclonal antibody” or “monoclonal antibody composition” as used herein refers to polypeptides, including antibodies, antibody fragments, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The phrase “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., (2000) J Mol Biol 296:57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948. The human antibodies of the disclosure may include amino acid residues not encoded by human sequences (e.g., mutations introduced by random or site-specific mutagenesis in vitro or by somatic mutation in vivo, or a conservative substitution to promote stability or manufacturing). The phrase “human monoclonal antibody” as used herein refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The phrase “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Specific binding between two entities means a binding with an equilibrium constant (KA) (kₒₙ/k_{off}) of at least 10² M-¹, at least 5×10² M⁻¹, at least 10³ M⁻¹, at least 5×10³ M⁻¹, at least 10⁴ M″ at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁶ M⁻¹, at least 10⁹ M⁻¹, at least 5×10⁹ M⁻¹, at least 10¹⁹ M⁻¹, at least 5×10¹⁹ M⁻¹, at least 10¹¹ M⁻¹, at least 5×10¹¹m-1, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴M⁻¹, at least 10¹⁵ M⁻¹, or at least 5×10¹⁵ M¹. The phrase “specifically (or selectively) binds” to an antibody (e.g., an IL-10 or IL-10R binding antibody) refers to a binding reaction that is determinative of the presence of a cognate antigen (e.g., a human IL-10 or IL-10R) in a heterogeneous population of proteins and other biologics. In addition to the equilibrium constant (KA) noted above, an IL-10 or IL-10R binding antibody of the disclosure typically also has a dissociation rate constant (KD) (k_{off}/kₒₙ) of less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁶ M, less than 10⁻⁸ M, less than 5×10⁻⁹ M, less than 10⁻⁹ M, less than 5×10⁻¹⁹ M, less than 10⁻¹⁰ m, less than 5×10⁻¹¹ M, less than 10⁻¹¹ M, less than 5×10⁻¹² M, less than 10⁻¹² m, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower, and binds to IL-10 or IL-10R with an affinity that is at least twofold greater than its affinity for binding to a non-specific antigen (e.g., HSA).

In one embodiment, the antibody or fragment thereof has dissociation constant (Ka) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM, less than 10 pM, less than 1 pM as assessed using a method described herein or known to one of skill in the art (e.g., a BIAcore assay, ELISA, FACS, SET) (Biacore International AB, Uppsala, Sweden). The term “ K_{aSSOC}” or “Kₐ”, as used herein, refers to the association rate of a particular antibody-antigen interaction, whereas the term “K0₈” or “K_{d},” as used herein, refers to the dissociation rate of a particular antibody-antigen interaction. The term “KD”, as used herein, refers to the dissociation constant, which is obtained from the ratio of Kj to Kₐ (i.e. Kj/Kₐ) and is expressed as a molar concentration (M). KD values for antibodies can be determined using methods well established in the art. A method for determining the KD of an antibody is by using surface plasmon resonance, or using a biosensor system such as a BIACORE system.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity. The term “avidity” as used herein refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

The term “valency” as used herein refers to the number of potential target binding sites in a polypeptide. Each target binding site specifically binds one target molecule or specific site (i.e, epitope) on a target molecule. When a polypeptide comprises more than one target binding site, each target binding site may specifically bind the same or different molecules (e.g., may bind to different molecules, e.g., different antigens, or different epitopes on the same molecule).

The phrase “antagonist antibody” as used herein refers to an antibody that binds with IL-10 or IL-10R and neutralizes the biological activity of IL-10 signaling, e.g., reduces, decreases and/or inhibits IL-10 induced signaling activity. Accordingly, an antibody that “inhibits” one or more of these IL-10 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (e.g., or when a control antibody of irrelevant specificity is present). An antibody that inhibits IL-10 activity effects such a statistically significant decrease by at least 10% of the measured parameter, by at least 50%>, 80%>or 90%>, and in certain embodiments an antibody may inhibit greater than 95%, 98% or 99% of IL-10 functional activity.

The phrase “isolated antibody” refers to an antibody that is substantially free of other antibodies having different antigenic specificities (e.g. an isolated antibody that specifically binds IL-10 or IL-10R is substantially free of antibodies that specifically bind antigens other than IL-10 or IL-10R). An isolated antibody that specifically binds IL-10 or IL-10R may, however, have cross-reactivity to other antigens. Moreover, an isolated antibody may be substantially free of other cellular material and/or chemicals.

The phrase “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

The terms “cross-compete” and “cross-competing” are used interchangeably herein to mean the ability of an antibody or other binding agent to interfere with the binding of other antibodies or binding agents to an IL-10 or IL-10R in a standard competitive binding assay.

The ability or extent to which an antibody or other binding agent is able to interfere with the binding of another antibody or binding molecule to IL-10 or IL-10R, and therefore whether it can be said to cross-compete according to the disclosure, can be determined using standard competition binding assays. One suitable assay involves the use of the BIACORE technology (e.g. by using the BIACORE 3000 instrument (Biacore, Uppsala, Sweden)), which can measure the extent of interactions using surface plasmon resonance technology. Another assay for measuring cross-competing uses an ELISA-based approach.

The term “optimized” as used herein refers to a nucleotide sequence has been altered to encode an amino acid sequence using codons that are preferred in the production cell or organism, generally a eukaryotic cell, for example, a cell of Pichia, a cell of Trichoderma, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

Standard assays to evaluate the binding ability of the antibodies toward IL-10 or IL-10R of various species are known in the art, including for example, ELISAs, western blots and RIAs. The binding kinetics (e.g., binding affinity) of the antibodies also can be assessed by standard assays known in the art, such as by BIACORE analysis, or FACS relative affinity (Scatchard). Assays to evaluate the effects of the antibodies on functional properties of IL-10 or IL-10R known in the art may be used.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The phrase “signal transduction” or “signaling activity” as used herein refers to a biochemical causal relationship generally initiated by a protein-protein interaction such as binding of a growth factor to a receptor, resulting in transmission of a signal from one portion of a cell to another portion of a cell.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

“Measuring” or “measurement” means assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “detecting” or “detection” may be used and is understood to cover all measuring or measurement as described herein.

The terms “sample” or “biological sample” as used herein, refers to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts. In some embodiments, the whole blood sample is further processed into serum or plasma samples.

“Treating”, “treat”, or “treatment” within the context of the instant disclosure, means an alleviation of symptoms associated with a disorder or disease, or halt of further progression or worsening of those symptoms, or prevention or prophylaxis of the disease or disorder.

### Pharmaceutical Compositions

To prepare pharmaceutical or sterile compositions including agents that inhibit IL-10 signaling, the agents may be mixed with a pharmaceutically acceptable carrier or excipient. In some embodiments, agents including IL-10 or IL-10R-binding antibodies (intact or binding fragments), the IL-10 or IL-10R-binding antibodies (intact or binding fragments) may be mixed with a pharmaceutically acceptable carrier or excipient. The compositions can additionally contain one or more other therapeutic agents that are suitable for treating or preventing diabetic ulcers.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al, (2003) New Engl. J. Med. 348:601-608; Milgrom et al, (1999) New Engl. J. Med. 341 : 1966-1973; Slamon et al, (2001) New Engl. J. Med. 344:783-792; Beniaminovitz et al, (2000) New Engl. J. Med. 342:613-619; Ghosh et al, (2003) New Engl. J. Med. 348:24-32; Lipsky et al, (2000) New Engl. J. Med. 343: 1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts. Compositions comprising antibodies or fragments thereof of the disclosure can be provided by continuous infusion, or by doses at intervals of, e.g., one day, one week, or 1-7 times per week. Doses may be provided topically, intravenously, subcutaneously, orally, nasally, rectally, intramuscular, intracerebrally, or by inhalation. A specific dose protocol is one involving the maximal dose or dose frequency that avoids significant undesirable side effects. A total weekly dose may be at least 0.05 μ/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 25 mg/kg, at least 30 mg/kg, at least 40 mg/kg or at least 50 mg/kg (see, e.g., Yang et al, (2003) New Engl. J. Med. 349:427-434; Herold et al, (2002) New Engl. J. Med. 346: 1692-1698; Liu et al, (1999) J. Neurol. Neurosurg. Psych. 67:451-456; Portielji et al, (2003) Cancer Immunol. Immunother. 52: 133-144). The desired dose of antibodies or fragments thereof is about the same as for an antibody or polypeptide, on a moles/kg body weight basis. The desired plasma concentration of the antibodies or fragments thereof is about, on a moles/kg body weight basis. The dose may be at least 15 μg at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg. The doses administered to a subject may number at least 1, 2, 3, 4, 5, 6, 7, 8, 9, 10, 11, or 12, or more. For antibodies or fragments thereof of the disclosure, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the antibodies or fragments thereof of the disclosure may be calculated using the patient's weight in kilograms (kg) multiplied by the dose to be administered in mg/kg. The dosage of the antibodies or fragments thereof of the disclosure may be 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight.

Unit dose of the antibodies or fragments thereof of the disclosure may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 60 mg, 0.25 mg to 40 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the antibodies or fragments thereof of the disclosure may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in a subject. Alternatively, the dosage of the antibodies or fragments thereof of the disclosure may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 .mu.g/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in the subject.

Doses of antibodies or fragments thereof of the disclosure may be repeated and the administrations may be separated by at least 1 day, 2 days, 3 days, 5 days, 7 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side effects (see, e.g., Maynard et al., (1996) A Handbook of SOPs for Good Clinical Practice, Interpharm Press, Boca Raton, Fla.; Dent (2001) Good Laboratory and Good Clinical Practice, Urch PubL, London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., (1983) Biopolymers 22:547-556; Langer et al., (1981) J. Biomed. Mater. Res. 15: 167-277; Langer (1982) Chem. Tech. 12:98-105; Epstein et al, (1985) Proc. Natl. Acad. Sci. USA 82:3688-3692; Hwang et al., (1980) Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the present disclosure may also be administered via one or more routes of administration using one or more of a variety of methods known in the art. As will be appreciated by the skilled artisan, the route and/or mode of administration will vary depending upon the desired results. Selected routes of administration for antibodies or fragments thereof of the disclosure include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-parenteral route, such as a topical, epidermal or mucosal route of administration, for example, intranasally, orally, vaginally, rectally, sublingually or topically. In one embodiment, the antibodies or fragments thereof of the disclosure is administered by infusion. In another embodiment, the multispecific epitope binding protein of the disclosure is administered subcutaneously. If the antibodies or fragments thereof of the disclosure are administered in a controlled release or sustained release system, a pump may be used to achieve controlled or sustained release (see Langer, supra; Sefton, (1987) CRC Crit. Ref Biomed. Eng. 14:20; Buchwald et al., (1980), Surgery 88:507; Saudek et al, (1989) N. Engl. J. Med. 321 :574). Polymeric materials can be used to achieve controlled or sustained release of the therapies of the disclosure (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al., (1985) Science 228: 190; During et al, (1989) Ann. Neurol. 25:351; Howard et al, (1989) J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer, (1990), Science 249: 1527- 1533). Any technique known to one of skill in the art can be used to produce sustained release formulations comprising one or more antibodies or fragments thereof of the disclosure. See, e.g., U.S. Pat. No. 4,526,938, PCT publication WO 91/05548, PCT publication WO 96/20698, Ning et al, (1996), Radiotherapy & Oncology 39: 179-189, Song et al, (1995) PDA Journal of Pharmaceutical Science & Technology 50:372-397, Cleek et al., (1997) Pro. Int'l Symp. Control. Rel. Bioact. Mater. 24:853-854, and Lam et al, (1997) Proc. Int'l Symp. Control Rel. Bioact. Mater. 24:759-760, each of which is incorporated herein by reference in their entirety.

If the antibodies or fragments thereof of the disclosure are administered topically, they can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising antibodies or fragments thereof are administered intranasally, it can be formulated in an aerosol form, spray, mist or in the form of drops. In particular, prophylactic or therapeutic agents for use according to the present disclosure can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%>; at least 40%>, or at least 50%.

Additional therapies (e.g., prophylactic or therapeutic agents), which can be administered in combination with the antibodies or fragments thereof of the disclosure may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibodies or fragments thereof of the disclosure. The two or more therapies may be administered within one same patient visit.

The antibodies or fragments thereof of the disclosure and the other therapies may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the antibodies or fragments thereof of the disclosure can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, (1989) J. Clin. Pharmacol. 29:685). Exemplary targeting moieties include folate or biotin (see, e.g., U.S. Pat. No. 5,416,016 to Low et al); mannosides (Umezawa et al, (1988) Biochem. Biophys. Res. Commun. 153: 1038); antibodies (Bloeman et al, (1995) FEBS Lett. 357: 140; Owais et al., (1995) Antimicrob. Agents Chemother. 39: 180); surfactant protein A receptor (Briscoe et al, (1995) Am. J. Physiol. 1233: 134); p 120 (Schreier et al, (1994) J. Biol. Chem. 269:9090); see also K. Keinanen; M. L. Laukkanen (1994) FEBS Lett. 346: 123; J. J. Killion; I. J. Fidler (1994) Immunomethods 4:273.

The disclosure provides protocols for the administration of pharmaceutical composition comprising antibodies or fragments thereof of the disclosure alone or in combination with other therapies to a subject in need thereof. The therapies (e.g., prophylactic or therapeutic agents) of the combination therapies of the present disclosure can be administered concomitantly or sequentially to a subject. The therapy (e.g., prophylactic or therapeutic agents) of the combination therapies of the present disclosure can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising antibodies or fragments thereof of the disclosure are administered to a subject in a sequence and within a time interval such that the antibodies of the disclosure can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

### EXAMPLES

### Results

Pro-inflammatory cytokine expression is substantially reduced during the acute phase of wound healing early after injury. CCL2 is one of several chemokines that can recruit monocytes into the wound and promote their differentiation into macrophages (Snyder et al., 2016). The fact that macrophage influx is substantially reduced in diabetic wounds early after injury (Wood et al., 2014) suggested that the expression of other pro-inflammatory cytokines may be similarly affected in these wounds early after injury. To gain insight into the dynamics of proinflammatory cytokine expression during wound healing, we first analyzed the expression profiles of IL-1β and TNF-α pro-inflammatory cytokines which are expressed early after injury but decline over time during normal wound healing (Hubner et al., 1996, Ridiandries et al., 2018). We generated full-thickness excisional wounds on the back of C57B (normal) and db/db (type 2 diabetic) mice as described (Goldufsky et al., 2015, Wood et al., 2014), and evaluated wound tissues (on days 1, 3, 6, and 10 post injury) for their IL-3β and TNF-α contents by ELISA. As expected, there was significant increases in IL-16 and TNF-α levels in C57B normal wounds early after injury, but they declined rapidly reaching their lowest levels in day 10 (FIG. 7A). In contrast and mirroring the macrophage response delay (Wood et al., 2014), IL-1β and TNF-α levels were significantly lower in db/db diabetic wounds early after injury but increased over time in older wounds. Supporting these data, the mRNA levels of IL-1β and TNF-α, (as assessed by RTPCR (after accounting for reduced macrophage response by normalizing the data to 18S), were also substantially reduced in day 1 db/db wounds as compared to normal wounds (FIG. 7B). Moreover, the expression of CCL2, CCL3, G-CSF, and GM-CSF (other cytokines produced early in wounds and shown to be important for wound healing (Dipietro et al., 2001, Huang et al., 2017, Mann et al., 2006, Ridiandries et al., 2018)), were also significantly reduced in day 1 db/db wounds as compared to C57B normal wounds, as assessed by mRNA analyses and Western blotting (FIG. 7C-E). Collectively, these data indicated that in complete contrast to diabetic chronic ulcers that are plagued with excessive and damaging pro-inflammatory cytokines (Pierce, 2001, Wetzler et al., 2000), diabetic wounds suffer from insufficient pro-inflammatory cytokines during the acute phase of wound healing early after injury. However, it remained unclear why proinflammatory cytokine production is dampened in diabetic wounds early after injury.

Toll-like receptor signaling is significantly diminished during the acute phase wound healing in diabetic wound. Toll-like receptors (TLRs) are major factories for the production of pro-inflammatory cytokines and play critical roles in wound healing (reviewed in (Chen and DiPietro, 2017, Kluwe et al., 2009, Macedo et al., 2007, Suga et al., 2014). Moreover, TLR4/MyD88 signaling has been shown to be required for the expression of CCL2 in Human monocytic cell line THP-1 in response to lipopolysaccharide (LPS) stimulation (Akhter et al., 2018). Although diabetic chronic ulcers show elevated TLR signaling (Dasu et al., 2010), little is known about TLR signaling in diabetic wound early after injury. We assessed the expression of representative TLRs (TLR1, TLR2, and TLR4) and representative components of TLR-mediated signaling (MyD88, TRAF6, and NF-kB (Kawasaki and Kawai, 2014)) in day 1 normal and diabetic wounds by Western blotting, after accounting for reduced macrophage response in these wounds by loading equal amounts of proteins. Data demonstrated that the expression of all indicated TLRs and TLR signaling components were substantially reduced in day 1 diabetic wound as compared to normal wound (FIG. 1A, and FIG. 8A). We further corroborated these data by mRNA analyses and histological assessments of TLR4 and MyD88, which showed significant reductions in their expressions in day 1 diabetic wound as compared to C57B normal wound (FIG. 1B-D). These data indicated that TLR signaling is significantly dampened in diabetic wound early after injury, thus providing a defective mechanism for insufficient pro-inflammatory cytokine production in diabetic wound early after injury. However, it remained unclear what was causing the reduction in the TLR signaling in diabetic wound early after injury.

Exposure to high glucose levels in diabetic range leads to impaired TLR signaling and reduced chemokine expression in monocytes. CCL2 expression is reduced in both type 1 and type 2 animals during the acute phase of wound healing and in monocytes extracted from type 1 diabetic individuals (Abke et al., 2006, Ishida et al., 2019, Wood et al., 2014), suggesting that exposure to high glucose may be responsible for this impairment. We purified human Peripheral Blood Monocytes (hPBMCs) and mouse Bone Marrow Derived Monocytes (mBMDMs) and exposed them to glucose levels in the normal range (90 mg/dl) or diabetic range (300 mg/dl) for 1 h. We then assessed the impact of high glucose on the expression of TLR signaling components (TLR2, TLR4, MyD88, and NF-kB (RelA, and RelB)) and pro-inflammatory cytokines (IL-1β, TNF-α, CCL2, and CCL3) by mRNA analysis using RT-PCR or by Western blotting. Exposure to high glucose for 1 h caused significant reductions in the mRNA levels of all indicated genes in hPBMCs and mBMDMs (FIG. 1E and FIG. 9C). Similarly, the corresponding proteins were also reduced in mBMDMs and hPBMCs, except MyD88 which was significantly reduced in mBMDMs but not in hPBMCs, suggesting that MyD88 protein stability/turnover may be differentially regulated in hPBMCs and mBMDMs (FIG. 1F and FIG. 9B, D, and E). Collectively, these data indicated that acute exposure to high glucose results in significant reduction in TLR-mediated signaling, leading to reduced pro-inflammatory cytokine production. However, the defective mechanism(s) underlying high glucose-induced reduction in TLR mediated signaling remained unknown.

IL-10 expression is significantly increased during the acute phase of healing in diabetic wounds and in response to short-term exposure to high glucose. IL-10 is a potent anti-inflammatory cytokine that inhibits TLR signaling and pro-inflammatory cytokine production through various mechanisms (Curtale et al., 2013, Knödler et al., 2009, Murray, 2005, Wang et al., 1995). Interestingly, IL-10 also inhibits glucose uptake in lipopolysaccharide (LPS)-stimulated macrophages (Ip et al., 2017). Moreover, exposure to high glucose has been shown to cause cellular damage, leading to pro-inflammatory responses (Devi et al., 2013, Park and Park, 2013). Taking these reports into consideration, we postulated that in diabetic environment where glucose is in excess, IL-10 may be upregulated as a protective measure to prevent excess glucose uptake in order to protect against glucose-induced cellular damage. Elevated IL-10 would in turn reduce TLR signaling and pro-inflammatory responses in monocytes through autocrine signaling.

To test our hypothesis, we first measured IL-10 levels in day 1 normal and diabetic wounds by ELISA. Data indicated that IL-10 levels were significantly higher in day 1 diabetic wounds as compared to normal wounds (FIG. 2A). IL-10 mRNA assessment by RT-PCR and protein level assessment by Western blotting corroborated the ELISA data, showing that IL-10 was significantly upregulated in day 1 diabetic wounds (FIG. 2B-D). Supporting these data, exposure to high glucose also caused significant increases in IL-10 mRNA and IL-10 protein levels in both cell lysates and supernatants of hPBMCs and mBMDMs (FIG. 2E-G and FIG. 9A-C). Importantly, glucose uptake, as determined by 2-NBDG fluorescent D-glucose analog, was significantly reduced in monocytes under high glucose (FIG. 2H), suggesting that increased IL-10 in the supernatant was dampening glucose uptake to mitigate cellular damage but also was inhibiting TLR signaling and pro-inflammatory cytokines production in an autocrine fashion.

Blocking IL-10 signaling enhances TLR signaling and pro-inflammatory cytokine production in monocytes and in diabetic wounds during the acute phase of healing. If IL-10 in the supernatant of monocytes exposed to high glucose is responsible for reducing glucose uptake and dampening TLR signaling and pro-inflammatory cytokine production in an autocrine fashion, then interfering with IL-10 signaling should reverse these effects in monocytes exposed to high glucose. To address this possibility, we exposed hPBMCs and mBMDMs to high glucose for 1 h in the presence of either an anti-IL-10 antibody (α-IL-10) or a control isoform antibody (α-IgG). Blocking IL-10 signaling reversed the deleterious effects of acute exposure to high glucose and increased glucose uptake (FIG. 3A), and increased TLR signaling and pro-inflammatory cytokine production, including CCL2 (FIG. 3B-D and FIG. 11). These results indicated that IL-10 autocrine signaling is responsible for reductions in glucose uptake, TLR signaling, and proinflammatory cytokine production in monocytes exposed to high glucose.

We next examined whether blocking IL-10 signaling would enhance TLR signaling and pro-inflammatory cytokine production in diabetic wound during the acute phase of healing. We treated db/db diabetic wounds topically with either α-IL-10 or α-IgG control isoform antibodies immediately after injury and assessed the expression of representative components of TLR signaling machinery in treated wounds at 2 h, 5 h, and 24 h (day 1) following treatment by Western blotting. Blocking IL-10 signaling by α-IL-10 antibody significantly increased the expression of indicated TLR components in diabetic wounds early after treatment, albeit with varying kinetics (FIG. 4A and FIG. 12). We corroborated these data by assessing the expression of representative TLR signaling components (TLR4 and MyD88) in day 1 wounds by mRNA analysis using RT-PCR, and by histological analysis using their specific antibodies (FIG. 4B-D).

To evaluate the long-term impact of α-IL-10 antibody treatment on pro-inflammatory cytokines production in diabetic wounds, we harvested the wound tissues at 2 h, 5 h, 24 h (Day 1), and Day 10 and assessed their IL-1E□, TNF-α, and CCL2 levels by ELISA. Diabetic wounds treated with α-IL-10 antibody showed significant increases in IL-3β and TNF-α early after treatment within the first 5 h, as compared to mock-treated db/db wounds, but significantly lower levels in day 10 older wounds (FIG. 4E-F). In contrast, IL-3β and TNF-α continued to rise in α-IgG treated db/db wounds (FIG. 4E-F). CCL2 expression showed similar pattern, except that it also remained higher in day 1 db/db wounds treated with α-IL-10 but by day 10, CCL2 expression also declined in these wounds as compared to mock-treated wounds (FIG. 4A).

Blocking IL-10 stimulates healing by jumpstarting TLR signaling and proinflammatory responses. We next assessed the impact of anti-IL-10 treatment on the dynamics of inflammatory leukocyte responses in diabetic wounds by immunohistochemistry, as described (Goldufsky et al., 2015, Wood et al., 2014) and in Materials & Methods. Leukocyte numbers were low in α-IgG treated diabetic wounds early after injury but continued to rise as these wounds aged, but in contrast, leukocyte numbers in α-IL10- treated diabetic wounds increased substantially early after injury but declined in day 10 wounds (FIG. 5A-B and FIG. 12).

Macrophages are generally classified as either classically activated pro-inflammatory macrophages (M1), or alternatively activated anti-inflammatory and reparative macrophages (M2) (Ferrante and Leibovich, 2012), although other M2 macrophage subtypes have also been described (Loegl et al., 2016). In normal healing, M1 macrophages predominate during the acute phase of healing early after injury and contribute to inflammatory responses, whereas M2 macrophages predominate the wound as it transitions into proliferation phase and they play a crucial role in healing (Krzyszczyk et al., 2018, Mirza et al., 2009). We assessed the impact of IL-10 blockage on the dynamics and distribution of M1 and M2 macrophages by co-staining the wound tissues with either F4/80 and M1 macrophage marker iNOS (inducible nitric oxide synthase), or F4/80 and M2 macrophage marker MMR/CD206 (macrophage mannose receptor) (Bastian et al., 2018, Klar et al., 2018). Data indicated that blocking IL-10 signaling by α-IL-10 antibody significantly increased both M1 and M2 macrophages during the acute phase of healing on days 1 and 3 (FIG. 5C-F and FIG. 13). However, M1 macrophages were substantially reduced in α-IL-10-treated day 10 diabetic wounds and the remaining macrophages in day 10 wounds were of the M2 type, as assessed by M1/M2 ratios (FIG. 5G).

We next assessed the impact of IL-10 signaling inhibition on wound healing in diabetic wound by digital photography. Treatments with α-IL-10 antibody resulted in significant healing in diabetic wounds (FIG. 6a-b). Consistent with these data, α-IL-10-treated diabetic wounds were substantially more re-epithelized and exhibited increased epidermal thickening (FIG. 6C-D and FIG. 14A for full wound images).

Fibroblast and myofibroblasts are key players in extracellular matrix production and granulation tissue maturation during the proliferation and the remodeling phases of wound healing (Cheng et al., 2016, Skalli et al., 1989, Wilgus et al., 2008). The persistent inflammatory environment in diabetic chronic ulcers has been shown to adversely affect fibroblast and myofibroblasts functions resulting in reduced collagen and elastin extracellular matrix deposition and impaired healing in diabetic chronic ulcers (Augustine et al., 2014, Diegelmann and Evans, 2004, Yue et al., 1986). We evaluated the impact of IL-10 inhibition on fibroblast, myofibroblast, collagen, elastin, and connective tissue regeneration in day 10 diabetic wounds, using their respective markers Vimentin, α-SMA, Elastin, and Masson's Trichrome staining (Goldufsky et al., 2015, Hinz, 2006, Wilgus et al., 2008). Diabetic wounds treated with α-IL-10 antibody showed substantial increases in all these healing markers (FIG. 6E-F and FIG. 14B). Collectively, these data indicated that diabetic wounds will not develop persistent non-resolving inflammation and heal properly, if IL-10 signaling is disrupted in them early after injury.

### Discussion

Paradoxically, diabetic wounds suffer from inadequate pro-inflammatory cytokine production and insufficient macrophage and leukocyte trafficking during the acute phase of healing early after injury (Ishida et al., 2019, Maruyama et al., 2007, Wood et al., 2014). Here we report, that acute exposure (1 h) to high glucose causes a rapid transformation of human and mouse monocytes into an anti-inflammatory phenotype, manifested by increased expression and secretion of IL-10 which in autocrine fashion, causes reduction in pro-inflammatory cytokine production by dampening TLR signaling in hPBMCs and mBMDMs.

We posit that the rapid rise in the IL-10 expression and secretion in response to acute exposure to high glucose is a protective measure taken by monocytes to reduce glucose uptake (as our data show) in order to protect themselves against cellular and organelle damage that increased cytoplasmic glucose has been reported to do (Allen et al., 2003, Kumar and Sitasawad, 2009, Vanhorebeek et al., 2009). Increased IL-10 would then transform monocytes into anti-inflammatory phenotype, by downregulating TLR signaling and pro-inflammatory cytokines' production.

There appears to be a disconnect between our data showing increased IL-10 expression and reduced pro-inflammatory cytokine production resulting from exposure to high glucose and the clinical reports indicating decreased IL-10 levels and increased pro-inflammatory cytokines in the serum of individuals with type 2 diabetes (Hotamisligil et al., 1995, Keane et al., 2017, Yaghini et al., 2011). We propose that despite initial increases in IL-10 to protect cells from damaging high glucose uptake, prolonged exposure to high glucose allows for gradual rise in cytosolic glucose level which eventually drives cells toward pro-inflammatory phenotype by shutting down IL-10 expression and by enhancing glycolysis and increasing oxidative stress, which are reported to be required for the induction of pro-inflammatory responses in macrophages (Freemerman et al., 2014, Ip et al., 2017). In this scenario, the blood environment in diabetes mirrors the chronic state of diabetic foot ulcer which is also plagued with persistent non-resolving inflammation (Bjarnsholt et al., 2008, Blakytny and Jude, 2006, Menke et al., 2007, Wetzler et al., 2000).

Wound macrophages primarily originate from circulating classical monocytes with a lifespan of ˜24h in circulation (Daley et al., 2010, Patel et al., 2017) and classical monocytes originate from bone marrow monocytes which are regenerated and released into the circulation within 1-3 hours, depending on injury and/or infection (Goto et al., 2003, Zhao et al., 2018). We propose that monocytes in the bone marrow of diabetic people or mice are not pro-inflammatory due to their short stay in that environment after their biogenesis. In contrast, monocytes in circulation are likely to display pro-inflammatory phenotype due to prolonged exposure to high glucose.

We further postulate that newly released monocytes from bone marrow, not the circulating monocytes, may be the monocytes migrating into the diabetic wound during the acute phase of healing. Our hypothesis is based on reports indicating monocytes (and neutrophils) extracted from the peripheral blood of diabetic individuals and diabetic mice exhibit many functional impairments (e.g, chemotaxis and bacterial killing), whereas these leukocytes appear to exhibit normal functions when they are extracted from the bone marrow of diabetic mice (Delamaire et al., 1997, Hill et al., 1983, Park et al., 2009, Repine et al., 1980, Scully et al., 2017, Sima et al., 1988) (Park et al., 2009).

Encouragingly, we found that one-time topical treatment with anti-IL-10 antibody significantly enhanced inflammatory responses during the acute phase of healing early after injury and substantially improved healing in diabetic wounds. Given that diabetic foot ulcers are already suffering from heightened inflammation, one might question the therapeutic value of anti-IL-10 therapies in stimulating wound healing and tissue repair in diabetic foot ulcers. We propose that anti-IL-10 directed treatments may have therapeutic potential in diabetic wound care, if applied topically after the surgical wound debridement process. Surgical debridement is performed as a standard of care weekly or biweekly in the clinics in order to reset a chronic non-healing wound into an acute wound (Cardinal et al., 2009, Golinko et al., 2008, Lebrun et al., 2010). Therefore, we propose that debrided wound environment is likely to be more similar to day 1 fresh wounds than to day 10 chronic wounds.

### MATERIALS AND METHODS

Animals: We have an approval from the Rush University Medical Center Institutional Animal Care and Use Committee (IACUC No: 18-037) to conduct research as indicated. All procedures complied strictly with the standards for care and use of animal subjects as stated in the Guide for the Care and Use of Laboratory Animals (Institute of Laboratory Animal Resources, National Academy of Sciences, Bethesda, MD, USA). We obtained 8-10 weeks old C57BL/6 (normal) and their diabetic littermates, C57BLKS-m leprdb(db/db) mice from the Jackson Laboratories (Bar Harbor, ME). These mice were allowed to acclimate to the environment for 1 week prior to experimentation. Wounding and wound infection were carried out as we described previously (Goldufsky et al., 2015, Wood et al., 2014).

MONOCYTES ISOLATION FROM HUMAN AND MOUSE: We have an Institutional Review Board (IRB)-approved protocol (ORA #: 16120704-IRB02) in accordance with the Common Rule (45CFR46, December 13, 2001) and any other governing regulations or subparts. This IRB-approved protocol allows us to collect blood samples from volunteers with their consents for these studies. Human monocytes from healthy subjects (both male and female) were purified from peripheral blood using the EasySep™ Human Monocytes Enrichment Kit (STEMCELL Technologies), according to manufacturer's protocol. Mouse monocytes were extracted from either bone marrow using the EasySep™ Mouse Monocytes Enrichment Kit (STEMCELL Technologies), as per manufacture's protocol. After isolation monocytes were either incubated for 1 hour or 24 hours with 90 mg/dL or 300 mg/dL glucose solution prepared in HBSS (Life Technologies) containing 2% HSA.

### Isolation of Macrophages

Peritoneal exudate cells from normal C57BL/6 and diabetic mice were harvested from peritoneal cavity using chilled serum-free RPMI 1640 medium and added to wells of 6-well tissue culture plates. After 2-h incubation at 37° C. in an atmosphere of 5% CO2 in air in a CO2 incubator, the non-adherent cells were removed by vigorous washing (three times) with warm serum-free RPMI 1640 medium and the adherent cells (1 x 106) were lysed for Western blotting. More than 95% of the adherent cell population was macrophages as determined by morphology and non-specific esterase staining (Feng et al., 2002, Tripathi and Sodhi, 2008).

HISTOPATHOLOGICAL EVALUATION: Leukocytes in wounds were identified by their rounded or polymorphonuclear morphology as described previously (Goldufsky et al., 2015, Kroin t al., 2016). Macrophages were assessed by immunohistochemical (IHC) analysis using F4/80 with iNOS (M1 macrophages) or F4/80 with MMR/CD206 (M2 macrophages) staining, as described (Lucas et al., 2010). Wound tissues' contents of IL-1β, TNF-α and CCL2 were assessed by ELISA, as described (Gupta et al., 2017). Wound healing was assessed by digital photography, by re-epithelization assessment using H&E staining, and by elastin, vimentin, α-smooth muscle actin (α-SMA) and collagen matrix deposition assessment using Masson's Trichrome staining, as described (Almine et al., 2012, Goldufsky et al., 2015, Wilgus et al., 2008, Wood et al., 2014).

GLUCOSE UPTAKE: Glucose uptake in monocytes was assessed by EZCell™ Glucose Uptake Assay Kit (Cat. No. K924 from BioVision) according to the manufacturer's protocol. Briefly, kit is based on using 2-NBDG fluorescent D-glucose uptake which is measured by using a set of enzymatic reactions that specifically oxidize glucose producing intermediates that react with the OxiRed™ Probe generating a fluorescence signal (Ex/Em=535/587 nm). The fluorescence signal is directly proportional to the amount of glucose that has been taken up and accumulated inside the cells. We were unable to assess the gradual increases in cellular glucose in monocytes exposed to prolonged high glucose (24 h) due to limitations with the 2-NBDG assay, which requires the use of hexokinase inhibitor to arrest glucose consumption after 2-NBDG addition and uptake. Given that hexokinase is the first enzyme in the glycolysis and glucose metabolism, its inhibition for 24 h would have severe consequences on cellular physiology and function.

WESTERN BLOTTING: We performed Western immunoblotting on cell lysates or on tissue lysates, using the indicated antibodies, as we described previously (Kroin et al., 2016, Shafikhani and Engel, 2006).

GENE EXPRESSION ANALYSIS BY REAL-TIME POLYMERASE CHAIN REACTION (RT-PCR): Gene expression at transcriptional level in wound tissues was assessed by mRNA analysis of indicated genes using RT-PCR, as described (Wood et al., 2014).

### Histopathological Evaluation

Wound healing was assessed by digital photography, by re-epithelization assessment using H&E staining, and by elastin, vimentin, α-smooth muscle actin (□-SMA) and collagen matrix deposition assessment using Masson's Trichrome staining, as described (Almine et al., 2012, Goldufsky et al., 2015, Wilgus et al., 2008, Wood et al., 2014). Leukocytes in wounds were identified by their rounded morphology, by polymorphonuclear morphology (in case of neutrophils), and by eosin positive staining (magenta/pink cytoplasm), as described previously (Goldufsky et al., 2015, Kroin et al., 2016). Macrophages trafficking into wounds were assessed by immunohistochemical (IHC) analysis using F4/80 with iNOS (M1 macrophages) or F4/80 with MMR/CD206 (M2 macrophages) staining (Lucas et al., 2010). The histological data, (obtained from n 5 mice/group and>9 random fields/wound/mouse), were normalized per wound surface area. Wound tissues' contents of IL-1β, TNF-α and CCL2 were assessed by ELISA (Gupta et al., 2017).

### Western Blot Analyses

Western immunoblotting was performed on cell lysates or on tissue lysates, using the indicated antibodies, as we described previously. Equal amounts of proteins (as determined by BSA analysis) were loaded (Kroin et al., 2016, Shafikhani and Engel, 2006).

### Gene Expression Analysis by Real-Time Polymerase Chain Reaction (RT-PCR)

After isolation, monocytes (hPBMCs and mBMDMs) were either incubated for 1 h with 90 mg/dL or 300 mg/dL glucose either in presence or absence of anti-IL-10 (5 μg/ml) or anti-IgG isotype (5 μg/ml) antibodies as indicated in the respective figure legends. After the completion of treatment incubation, total RNA was isolated using Trizol method, followed by cDNA preparation and RTPCR analysis. Similarly, gene expression at transcriptional level in wound tissues was assessed by mRNA analysis of indicated genes using RT-PCR, as we described (Wood et al., 2014). Briefly, cDNA was generated using SuperScript™ III First-Strand Synthesis System cDNA Synthesis Kit from Thermo Fisher, according to manufacturer's protocol. RT-PCR was then performed with gene-specific primer pairs mentioned in the “Key Resources Table”, using the Applied Biosystems QuantStudio™ 7 Flex Real-Time PCR System. The data were calculated using the 2-ΔΔCt method and were presented as ratio of transcripts for gene of interest normalized to GAPDH or 18S.

QUANTIFICATION AND STATISTICAL ANALYSIS: Statistical analyses were performed by One-way analysis of variance, or by Student t-test, using the GraphPad Prism software. Data are presented as mean±SEM. P-values less than or equal to 0.05 were taken as significant. All statistical details of experiments can be found in the figure legends including the statistical tests used with number of replicates, mean and SEM.

TABLE I
Reagents

| | | |
|---|---|---|
| REAGENT or RESOURCE | SOURCE | IDENTIFIER |

Antibodies

| | | |
|---|---|---|
| NF-κB p65 (D14E12) XP ® Rabbit mAb | Cell Signaling Technology | Cat. No. 8242 |
| IL-1β (3A6) Mouse mAb | Cell Signaling Technology | Cat. No. 12242 |
| F4/80 (D2S9R) XP ® Rabbit mAb | Cell Signaling Technology | Cat. No. 70076 |
| Mouse (G3A1) mAb IgG1 Isotype Control | Cell Signaling Technology | Cat. No. 5415 |
| GAPDH Antibody Rabbit Polyclonal | Proteintech | Cat. No. 1094-I-AP |
| MCP-1 Antibody (Mouse Specific) | Cell Signaling Technology | Cat. No. 2029 |
| TLR1 Rat anti-Mouse, Clone: 285923 | R &D systems | Cat. No. MAB1475 |
| TLR2 Goat anti-Mouse, Polyclonal | R &D systems | Cat. No. AF1530 |
| TLR4 Antibody (25) | Santa Cruz Biotechnology | Cat. No. 293072 |
| IL-10 Antibody (E-10) | Santa Cruz Biotechnology | Cat. No. 8438 |
| TLR2 Antibody (TL2.1) | Santa Cruz Biotechnology | Cat. No. 21759 |
| TNF alpha Antibody | Santa Cruz Biotechnology | Cat. No. 52746 |
| Anti-MyD88 antibody | Abcam | Cat. No. ab135693 |
| Anti-IL-10 antibody [JES5-2A5] | Abcam | Cat. No. ab33471 |
| Recombinant Anti-G-CSF antibody | Abcam | Cat. No. ab181053 |
| [EPR3203(N)(B)] | | |
| TRAF6 Polyclonal Antibody | Invitrogen | Cat. No. 38-0900 |
| CCL3 Recombinant Rabbit Monoclonal | Invitrogen | Cat. No. 701097 |
| Antibody (1H20L19) | | |
| CCL3 Monoclonal Antibody (756613) | Invitrogen | Cat. No. MA5-24364 |
| GM-CSF Monoclonal Antibody (22E9) | Invitrogen | Cat. No. MM500C |
| IL-10 Monoclonal Antibody (JES3-9D7) | Invitrogen | Cat. No. AHC0102 |
| Mouse (MOPC-21) mAb IgG1 Isotype | Cell Signaling Technology | Cat. No. 4097 |
| Control | | |
| InVivoMAb anti-mouse IL-10, Clone: | InVivoMab Antibodies | Cat. No. BE0049 |
| JES5-2A5 | | |
| anti-horseradish peroxidase, Clone: | InVivoMab Antibodies | Cat. No. BE0088 |
| HRPN | | |
| F4/80 (D2S9R) XP ® Rabbit mAb | Cell Signaling Technology | Cat. No. 70076 |
| MMR/CD206/Mannose Receptor | Novus Biologicals | Cat no. NBP1-90020 |
| Antibody | | |
| iNOS Antibody | Novus Biologicals | Cat no. NBP2-22119 |
| F4/80 Antibody (C-7) | Santa Cruz Biotechnology | Cat no. 377009 |
| Goat anti-Rabbit IgG (H+L) Cross- | Thermo Fisher | Cat no. A11-012 |
| Adsorbed Secondary Antibody, Alexa | | |
| Fluor 594 | | |
| Alexa Fluor ® 488 AffiniPure Goat Anti- | Jackson Immuno Research | Cat no. 115-545-062 |
| Mouse IgG (H+L) | | |
| Anti-a-SMA antibody | Abcam | Cat. No. ab5694 |
| Anti-vimentin antibody | Abcam | Cat. No. ab92547 |
| Anti-IL-10R antibody | BioLegend | Cat. No. 112705 |
| Anti-IL-10R antibody | Bio X Cell | Cat. No. BE0050 |

Reagents and kits

| | | |
|---|---|---|
| Hematoxylin | Thermo Fisher | Cat. No. 7111L |
| Eosin Y | Thermo Fisher | Cat. No. 7211L |
| Bluing Reagent | Thermo Fisher | Cat. No. 7301L |
| Masson's Trichrome stain | Abcam | Cat. No. ab150686 |
| EasySep Human Monocytes Enrichment Kit | STEMCELL Technologies | Cat. No. 19359 |
| EasySep Mouse monocytes Enrichment Kit | STEMCELL Technologies | Cat. No. 19861 |
| EasySep Buffer | STEMCELL Technologies | Cat. No. 20144 |
| SYBR ™ Green PCR Master Mix | Thermo Fisher | Cat. No. 4309155 |
| CCL3 Monoclonal Antibody (756613) | Invitrogen | Cat. No. MA5-24364 |
| GM-CSF Monoclonal Antibody (22E9) | Invitrogen | Cat. No. MM500C |
| IL-10 Monoclonal Antibody (JES3-9D7) | Invitrogen | Cat. No. AHC0102 |
| Mouse (MOPC-21) mAb IgG1 Isotype | Cell Signaling Technology | Cat. No. 4097 |
| Control | | |
| InVivoMAb anti-mouse IL-10, Clone: JES5- | InVivoMab Antibodies | Cat. No. BE0049 |
| 2A5 | | |
| anti-horseradish peroxidase, Clone: HRPN | InVivoMab Antibodies | Cat. No. BE0088 |
| F4/80 (D2S9R) XP ® Rabbit mAb | Cell Signaling Technology | Cat. No. 70076 |
| MMR/CD206/Mannose Receptor Antibody | Novus Biologicals | Cat no. NBP1-90020 |
| iNOS Antibody | Novus Biologicals | Cat no. NBP2-22119 |
| F4/80 Antibody (C-7) | Santa Cruz Biotechnology | Cat no. 377009 |
| Goat anti-Rabbit IgG (H+L) Cross-Adsorbed | Thermo Fisher | Cat no. A11-012 |
| Secondary Antibody, Alexa Fluor 594 | | |
| Alexa Fluor ® 488 AffiniPure Goat Anti- | Jackson Immuno Research | Cat no. 115-545-062 |
| Mouse IgG (H+L) | | |
| Anti-α-SMA antibody | Abcam | Cat. No. ab5694 |
| Anti-vimentin antibody | Abcam | Cat. No. ab92547 |

Reagents and kits

| | | |
|---|---|---|
| Hematoxylin | Thermo Fisher | Cat. No. 7111L |
| Eosin Y | Thermo Fisher | Cat. No. 7211L |
| Bluing Reagent | Thermo Fisher | Cat. No. 7301L |
| Masson's Trichrome stain | Abcam | Cat. No. ab150686 |
| EasySep Human Monocytes Enrichment Kit | STEMCELL Technologies | Cat. No. 19359 |
| EasySep Mouse monocytes Enrichment Kit | STEMCELL Technologies | Cat. No. 19861 |
| EasySep Buffer | STEMCELL Technologies | Cat. No. 20144 |
| SYBR ™ Green PCR Master Mix | Thermo Fisher | Cat. No. 4309155 |
| SuperScript ™ III First-Strand Synthesis | Thermo Fisher | Cat. No. 18080051 |
| System | | |
| Elastic connective tissue stain | Abcam | Cat. No. ab150667 |

Commercial Assays

CCL2 ELISA kit
IL-1b ELISA kit
TNF-a ELISA kit
EZCell ™ Glucose Uptake Assay kit

Experimental Models: Organisms

Mouse: C57BL/6J
Mouse: C57BLKS-m leprdb(db/db)

Oligonucleotides
Human:

| | | |
|---|---|---|
| Tlr2 Forward: | Integrated DNA | N/A |
| GAAGAGTGAGTGGTGCAAGTAT, (SEQ | Technologies | |
| ID NO: 1) | | |
| Reverse: | | |
| AATGGGCTCCAGAAGAATGAG; (SEQ ID | | |
| NO: 2) | | |
| Tlr4 Forward: | Integrated DNA | N/A |
| TTTCAGCTCTGCCTTCACTAC, (SEQ ID | Technologies | |
| NO: 3) | | |
| Reverse: | | |
| GACACCACAACAATCACCTTTC (SEQ | | |
| ID NO: 4) | | |
| MYD88 Forward: | Integrated DNA | N/A |
| CTGTGTCTGGTCTATTGCTAGTG, (SEQ | Technologies | |
| ID NO: 5) | | |
| Reverse: TTCCTTGCTCTGCAGGTAATC | | |
| (SEQ ID NO: 6) | | |
| IL1 Forward: | Integrated DNA | N/A |
| CAAAGGCGGCCAGGATATAA, (SEQ ID | Technologies | |
| NO: 7) | | |
| Reverse: | | |
| CTAGGGATTGAGTCCACATTCAG (SEQ | | |
| ID NO: 8) | | |
| TNF Forward: | Integrated DNA | N/A |
| GCAGGTCTACTTTGGGATCATT, (SEQ | Technologies | |
| ID NO: 9) | | |
| Reverse: AGAAGAGGTTGAGGGTGTC | | |
| (SEQ ID NO: 10) | | |
| RelA Forward: | Integrated DNA | N/A |
| CTGTCCTTTCTCATCCCATCTT, (SEQ ID | Technologies | |
| NO: 11) | | |
| Reverse: TCCTCTTTCTGCACCTTGTC | | |
| (SEQ ID NO: 12) | | |
| RelB Forward: | Integrated DNA | N/A |
| CTGCGGATTTGCCGAATTAAC, (SEQ ID | Technologies | |
| NO: 13) | | |
| Reverse: | | |
| ACACCACTGATATGTCCTCTTTC (SEQ | | |
| ID NO: 14) | | |
| Ccl2 Forward: | Integrated DNA | N/A |
| TCATAGCAGCCACCTTCATTC, (SEQ ID | Technologies | |
| NO: 15) | | |
| Reverse: | | |
| CTCTGCACTGAGATCTTCCTATTG (SEQ | | |
| ID NO: 16) | | |
| Ccl3 Forward: | Integrated DNA | N/A |
| GGCAGATTCCACAGAATTTCATAG, | Technologies | |
| (SEQ ID NO: 17) | | |
| Reverse: TCGCTTGGTTAGGAAGATGAC | | |
| (SEQ ID NO: 18) | | |
| IL10 Forward: | Integrated DNA | N/A |
| TCCTTGCTGGAGGACTTTAAGGGT, | Technologies | |
| (SEQ ID NO: 19) | | |
| Reverse: | | |
| TGTCTGGGTCTTGGTTCTCAGCTT | | |
| (SEQ ID NO: 20) | | |
| 18s Forward: | Integrated DNA | N/A |
| CACGGACAGGATTGACAGATT, (SEQ ID | Technologies | |
| NO: 21) | | |
| Reverse: GCCAGAGTCTCGTTCGTTATC | | |
| (SEQ ID NO: 22) | | |
| GAPDH Forward: | Integrated DNA | N/A |
| GGTGTGAACCATGAGAAGTATGA, (SEQ | Technologies | |
| ID NO: 23) | | |
| Reverse: | | |
| GAGTCCTTCCACGATACCAAAG (SEQ | | |
| ID NO: 24) | | |

Mice:

| | | |
|---|---|---|
| granulocyte (Csf3) | Integrated DNA | N/A |
| Forward: TGTTCCCAAACTGGGTTCTT, | Technologies | |
| (SEQ ID NO: 25) | | |
| Reverse: TGGCTGCCACTGTTTCTT | | |
| (SEQ ID NO: 26) | | |
| GM-CSF | Integrated DNA | N/A |
| Forward: AGCTCTGAATCCAGCTTCTC, | Technologies | |
| (SEQ ID NO: 27) | | |
| Reverse: CCACATCTCTTGGTCCCTTTA | | |
| (SEQ ID NO: 28) | | |
| Ccl2 Forward: | Integrated DNA | N/A |
| CTCACCTGCTGCTACTCATTC, (SEQ ID | Technologies | |
| NO: 29) | | |
| Reverse: ACTACAGCTTCTTTGGGACAC | | |
| (SEQ ID NO: 30) | | |
| Ccl3 | Integrated DNA | N/A |
| Forward: | Technologies | |
| TCACTGACCTGGAACTGAATG, (SEQ ID | | |
| NO: 31) | | |
| Reverse: CAGCTTATAGGAGATGGAGCT | | |
| ATG (SEQ ID NO: 32) | | |
| Rela Forward: | Integrated DNA | N/A |
| CCGACTTGTTTGGGTGATCT, (SEQ ID | Technologies | |
| NO: 33) | | |
| Reverse: TCCGTCTCCAGGAGGTTAAT | | |
| (SEQ ID NO: 34) | | |
| Relb Forward: | Integrated DNA | N/A |
| TGCCGAATCAACAAGGAGAG, (SEQ ID | technologies | |
| NO: 35) | | |
| Reverse: TGCTGAACACCACGGATATG | | |
| (SEQ ID NO: 36) | | |
| Tlr2 Forward: | Integrated DNA | N/A |
| CACTATCCGGAGGTTGCATATC, (SEQ | Technologies | |
| ID NO: 37) | | |
| Reverse: | | |
| GGAAGACCTTGCTGTTCTCTAC (SEQ | | |
| ID NO: 38) | | |
| Tlr4 | Integrated DNA | N/A |
| Forward: GAGCAAACAGCAGAGGAAGA, | Technologies | |
| (SEQ ID NO: 39) | | |
| Reverse: | | |
| CCAGGTGAGCTGTAGCATTTA (SEQ ID | | |
| NO: 40) | | |
| Myd88 | Integrated DNA | N/A |
| Forward: AGCAACTAGGACTGCCTTTC, | Technologies | |
| (SEQ ID NO: 41) | | |
| Reverse: | | |
| GAACTCTTCCACTCAGCTATCC (SEQ | | |
| ID NO: 42) | | |
| IL-1 Forward: | Integrated DNA | N/A |
| GCACTACAGGCTCCGAGATGAAC; | Technologies | |
| (SEQ ID NO: 43) | | |
| Reverse: | | |
| TTGTCGTTGCTTGGTTCTCCTTGT (SEQ | | |
| ID NO: 44) | | |
| Tnf Forward: | Integrated DNA | N/A |
| TTGTCTACTCCCAGGTTCTCT, (SEQ ID | Technologies | |
| NO: 45) | | |
| Reverse: | | |
| GAGGTTGACTTTCTCCTGGTATG (SEQ | | |
| ID NO: 46) | | |
| GAPDH Forward: | Integrated DNA | N/A |
| TTGGGTTGTACATCCAAGCA, (SEQ ID | Technologies | |
| NO: 47) | | |
| Reverse: CAAGAAACAGGGGAGCTGAG | | |
| (SEQ ID NO: 48) | | |

Software and Algorithms

| | | |
|---|---|---|
| GraphPad Prism | GraphPad | https://graphpad.com/ |
| | | scientific- |
| | | software/prism/ |

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A method of treating a wound site in a diabetic patient comprising administering a therapeutically effective amount of an agent that inhibits Interleukin-10 (IL-10) signaling to the wound site.

2. The method according to claim 1, wherein the diabetic patient has type II or adult-onset diabetes.

3. The method according to claim 1, comprising administering the therapeutically effective amount of the agent within about one day of appearance of the wound site.

4. The method according to claim 1, comprising debriding the wound site and administering the therapeutically effective amount of the agent.

5. The method according to claim 1, wherein the agent antagonizes IL-10 or IL-10 Receptor (IL-10R) to inhibit IL-10 signaling.

6. The method according to claim 1, wherein the agent is selected from the group consisting of a nucleic acid, small molecule, peptide, or polypeptide.

7. The method according to claim 6, wherein the agent is a polypeptide comprising an antibody or a fragment thereof that specifically binds to IL-10 or IL-10R.

8. The method according to claim 7, wherein the antibody or fragment thereof is humanized.

9. The method according to claim 1, comprising topically administering the therapeutically effective amount of the agent that inhibits IL-10 signaling.

10. The method according to claim 1, comprising administering the therapeutically effective amount of the agent to the wound site to restore a macrophage response at the wound site.

11. The method according to claim 1, wherein the wound site is not infected.

12. The method according to claim 1, wherein the wound site is infected.
